(19) United States
(12) Patent Application Publication
(10) Pub. No.: US 2019/0365489 A1
(43) Pub. Date: Dec. 5, 2019

(54) MEDICAL SUPPORT ARM SYSTEM AND CONTROL DEVICE

(71) Applicant: SONY CORPORATION, TOKYO (JP)

(72) Inventor: TAKARA KASAI, KANAGAWA (JP)

(21) Appl. No.: 16/485,587

(22) PCT Filed: Feb. 16, 2018
(86) PCT No.: PCT/JP2018/005594

§ 371 Date: Aug. 13, 2019

(30) Foreign Application Priority Data
Feb. 28, 2017 (JP) 2017-035959

(51) Int. Cl. A61B 34/30; G01L 5/00; B25J 13/08; B25J 15/00; A61B 90/00; A61B 1/00; A61B 1/055; A61B 1/06; A61B 1/05; A61B 90/25; A61B 90/50

(52) U.S. Cl. CPC A61B 34/30; A61B 2034/301; B25J 13/085; B25J 15/0019; A61B 90/06; A61B 1/00149; A61B 1/055; A61B 1/0676; A61B 1/05; A61B 1/00006; A61B 90/25; A61B 90/50; A61B 2090/064; A61B 2090/066; G01L 5/00

(57) ABSTRACT

Provision of a technology capable of realizing estimation of forces of various types of disturbance acting on a robot arm in a surgical environment is desirable. Provided is a medical support arm system including a joint state acquisition unit configured to acquire a state of a joint unit of an arm unit, and an external force estimation unit configured to estimate an external force due to predetermined disturbance on the basis of a condition that the external force due to the predetermined disturbance is limited to one predetermined direction or a plurality of predetermined directions, and a state of the joint unit.

### TECHNICAL FIELD

The present disclosure relates to a medical support arm system and a control device.

### BACKGROUND ART

Conventionally, for example, Patent Document 1 describes, in a medical observation device, a configuration including an imaging unit that captures an image of an operation site, and a holding unit to which the imaging unit is connected and provided with rotation axes in an operable manner with at least six degrees of freedom, in which at least two axes, of the rotation axes, are active axes controlled to be driven on the basis of states of the rotation axes, and at least one axis, of the rotation axes, is a passive axis rotated according to a direct operation with contact from an outside.

### CITATION LIST

### Patent Document

Patent Document 1: International Publication No. 2016/017532

### SUMMARY OF THE INVENTION

### Problems to be Solved by the Invention

By the way, an arm intended for surgery support is used in an environment where various types of disturbance act. However, it is generally difficult to estimate the force acting from the disturbance regardless of conditions such as an environment and a scene.

Therefore, provision of a technology capable of realizing estimation of forces of various types of disturbance acting on an arm in a surgical environment is desirable.

### Solutions to Problems

According to the present disclosure, provided is a medical support arm system including a joint state acquisition unit configured to acquire a state of a joint unit of an arm unit, and an external force estimation unit configured to estimate an external force due to predetermined disturbance on the basis of a condition that the external force due to the predetermined disturbance is limited to one predetermined direction or a plurality of predetermined directions, and a state of the joint unit.

### Effects of the Invention

According to the above-described present disclosure, estimation of forces of various types of disturbance acting on an arm in a surgical environment can be realized.

Note that the above-described effect is not necessarily limited, and any of effects described in the present specification or other effects that can be grasped from the present specification may be exerted in addition to or in place of the above-described effect.

### BRIEF DESCRIPTION OF DRAWINGS

FIG. 1 is a diagram illustrating an example of a schematic configuration of an endoscopic surgical system to which the technology according to the present disclosure is applicable.

FIG. 2 is a block diagram illustrating an example of functional configurations of a camera head and a CCU illustrated in FIG. 1.

FIG. 3 is a perspective view illustrating a configuration example of a medical support arm device according to an embodiment of the present disclosure.

FIG. 4 is an explanatory diagram for describing ideal joint control according to an embodiment of the present disclosure.

FIG. 5 is a functional block diagram illustrating a configuration example of an arm control system according to an embodiment of the present disclosure.

FIG. 6 is a view illustrating an example of a schematic configuration of a microsurgical system to which the technology according to the present disclosure is applicable.

FIG. 7 is a view illustrating an appearance of a hard endoscope unit.

FIG. 8 is an enlarged view of a connection portion.

FIG. 9 is a view for describing an example of a force acting from a trocar point.

FIG. 10 is a view for describing an example of joint control in a case where an observation point is placed at a distal end of the hard endoscope.

FIG. 11 is a view for describing an example of joint control in a case where an observation point is placed at a distal end of the hard endoscope.

FIG. 12 is a diagram illustrating a specific configuration example of the arm control system.

### MODE FOR CARRYING OUT THE INVENTION

Favorable embodiments of the present disclosure will be described in detail with reference to the appended drawings. Note that, in the present specification and drawings, redundant description of configuration elements having substantially the same functional configuration is omitted by providing the same sign.

Note that the description will be given in the following order.

1. Configuration Example of Endoscopic System

2. Specific Configuration Example of Support Arm Device

3. Example of configuration of Microsurgical System

4. Estimation of Force Acting from Disturbance According to Present Embodiment

5. Joint Control According to External Force According to Present Embodiment

6. Specific Configuration Example of Arm Control System

7. Conclusion

### 1. Configuration Example of Endoscopic System

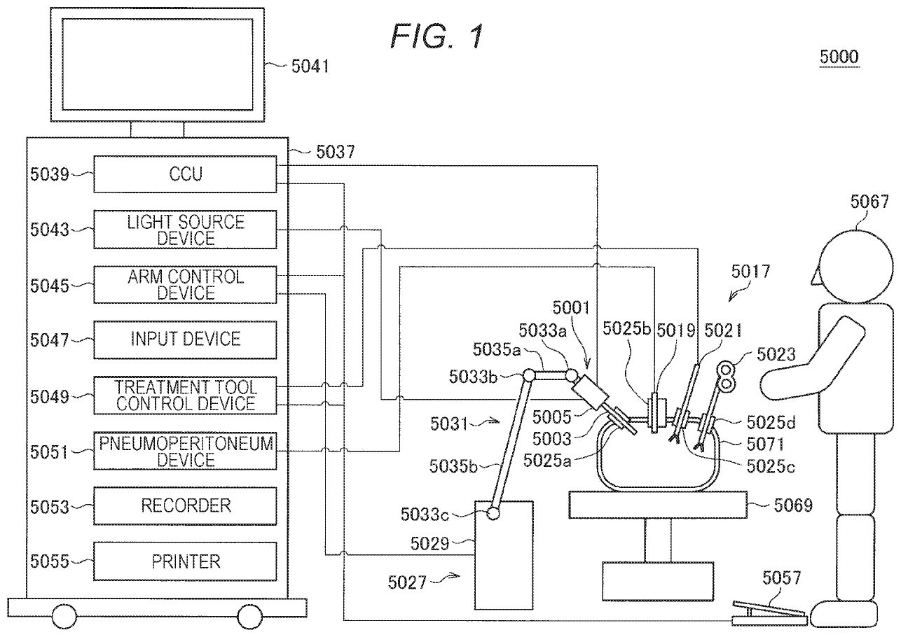

FIG. 1 is a diagram illustrating an example of a schematic configuration of an endoscopic surgical system 5000 to which the technology according to the present disclosure is applicable. FIG. 1 illustrates a state in which an operator (surgeon) 5067 is performing an operation on a patient 5071 on a patient bed 5069, using the endoscopic surgical system 5000. As illustrated, the endoscopic surgical system 5000 includes an endoscope 5001, other surgical tools 5017, a support arm device 5027 that supports the endoscope 5001, and a cart 5037 in which various devices for endoscopic surgery are mounted.

In an endoscopic surgery, a plurality of cylindrical puncture instruments called trocars 5025a to 5025d is punctured into an abdominal wall instead of cutting the abdominal wall and opening the abdomen. Then, a lens barrel 5003 of the endoscope 5001 and other surgical tools 5017 are inserted into a body cavity of the patient 5071 through the trocars 5025a to 5025d. In the illustrated example, as the other surgical tools 5017, a pneumoperitoneum tube 5019, an energy treatment tool 5021, and a forceps 5023 are inserted into the body cavity of the patient 5071. Furthermore, the energy treatment tool 5021 is a treatment tool for performing incision and detachment of tissue, sealing of a blood vessel, and the like with a high-frequency current or an ultrasonic vibration. Note that the illustrated surgical tools 5017 are mere examples, and various kinds of surgical tools typically used in endoscopic surgery such as tweezers and a retractor may be used as the surgical tool 5017.

An image of an operation site in the body cavity of the patient 5071 captured by the endoscope 5001 is displayed on a display device 5041. The operator 5067 performs treatment such as removal of an affected part, using the energy treatment tool 5021 and the forceps 5023 while viewing the image of the operation site displayed on the display device 5041 in real time. Note that the pneumoperitoneum tube 5019, the energy treatment tool 5021, and the forceps 5023 are supported by the operator 5067, an assistant, or the like during surgery, although illustration is omitted.

(Support Arm Device)

The support arm device 5027 includes an arm unit 5031 extending from a base unit 5029. In the illustrated example, the arm unit 5031 includes joint units 5033a, 5033b, and 5033c, and links 5035a and 5035b, and is driven under the control of an arm control device 5045. The endoscope 5001 is supported by the arm unit 5031, and the position and posture of the endoscope 5001 are controlled. With the control, stable fixation of the position of the endoscope 5001 can be realized.

(Endoscope)

The endoscope 5001 includes the lens barrel 5003 and a camera head 5005. A region having a predetermined length from a distal end of the lens barrel 5003 is inserted into the body cavity of the patient 5071. The camera head 5005 is connected to a proximal end of the lens barrel 5003. In the illustrated example, the endoscope 5001 configured as a so-called hard endoscope including the hard lens barrel 5003 is illustrated.

However, the endoscope 5001 may be configured as a so-called soft endoscope including the soft lens barrel 5003.

An opening portion in which an object lens is fit is provided in the distal end of the lens barrel 5003. A light source device 5043 is connected to the endoscope 5001, and light generated by the light source device 5043 is guided to the distal end of the lens barrel 5003 by a light guide extending inside the lens barrel 5003 and an observation target in the body cavity of the patient 5071 is irradiated with the light through the object lens. Note that the endoscope 5001 may be a direct-viewing endoscope, an oblique-viewing endoscope, or a side-viewing endoscope.

An optical system and an imaging element are provided inside the camera head 5005, and reflected light (observation light) from the observation target is condensed to the imaging element by the optical system. The observation light is photoelectrically converted by the imaging element, and an electrical signal corresponding to the observation light, that is, an image signal corresponding to an observed image is generated. The image signal is transmitted to a camera control unit (CCU) 5039 as raw data. Note that the camera head 5005 has a function to adjust magnification and a focal length by appropriately driving the optical system.

Note that a plurality of the imaging elements may be provided in the camera head 5005 to support three-dimensional (3D) display, and the like, for example. In this case, a plurality of relay optical systems is provided inside the lens barrel 5003 to guide the observation light to each of the plurality of imaging elements.

(Various Devices Mounted in Cart)

The CCU 5039 includes a central processing unit (CPU), a graphics processing unit (GPU), and the like, and centrally controls the operation of the endoscope 5001 and the display device 5041. Specifically, the CCU 5039 receives the image signal from the camera head 5005, and applies various types of image processing for displaying an image based on the image signal, such as developing processing (demosaicing processing), for example, to the image signal. The CCU 5039 provides the image signal to which the image processing has been applied to the display device 5041. Furthermore, the CCU 5039 transmits a control signal to the camera head 5005 to control its driving. The control signal may include information regarding imaging conditions such as the magnification and focal length.

The display device 5041 displays an image based on the image signal to which the image processing has been applied by the CCU 5039, under the control of the CCU 5039. In a case where the endoscope 5001 supports high-resolution capturing such as 4K (horizontal pixel number 3840×vertical pixel number 2160) or 8K (horizontal pixel number 7680×vertical pixel number 4320), and/or in a case where the endoscope 5001 supports 3D display, for example, the display device 5041, which can perform high-resolution display and/or 3D display, can be used corresponding to each case. In a case where the endoscope 5001 supports the high-resolution capturing such as 4K or 8K, a greater sense of immersion can be obtained by use of the display device 5041 with the size of 55 inches or more. Furthermore, a plurality of display devices 5041 having different resolutions and sizes may be provided depending on the application.

The light source device 5043 includes a light source such as a light emitting diode (LED) for example, and supplies irradiation light to the endoscope 5001 in capturing an operation portion.

The arm control device 5045 includes a processor such as a CPU, and is operated according to a predetermined program, thereby to control driving of the arm unit 5031 of the support arm device 5027 according to a predetermined control method.

An input device 5047 is an input interface for the endoscopic surgical system 5000. The user can input various types of information and instructions to the endoscopic surgical system 5000 through the input device 5047. For example, the user inputs various types of information regarding surgery, such as patient's physical information and information of an operative procedure of the surgery, through the input device 5047. Furthermore, for example, the user inputs an instruction to drive the arm unit 5031, an instruction to change the imaging conditions (such as the type of the irradiation light, the magnification, and the focal length) of the endoscope 5001, an instruction to drive the energy treatment tool 5021, or the like through the input device 5047.

The type of the input device 5047 is not limited, and the input device 5047 may be one of various known input devices. For example, a mouse, a keyboard, a touch panel, a switch, a foot switch 5057, and/or a lever can be applied to the input device 5047. In a case where a touch panel is used as the input device 5047, the touch panel may be provided on a display surface of the display device 5041.

Alternatively, the input device 5047 is a device worn by the user, such as a glass-type wearable device or a head mounted display (HMD), for example, and various inputs are performed according to a gesture or a line of sight of the user detected by the device. Furthermore, the input device 5047 includes a camera capable of detecting a movement of the user, and various inputs are performed according to a gesture or a line of sight of the user detected from a video captured by the camera. Moreover, the input device 5047 includes a microphone capable of collecting a voice of the user, and various inputs are performed by an audio through the microphone. In this way, the input device 5047 is configured to be able to input various types of information in a non-contact manner, whereby the user (for example, the operator 5067) in particular belonging to a clean area can operate a device belonging to a filthy area in a non-contact manner. Furthermore, since the user can operate the device without releasing his/her hand from the possessed surgical tool, the user's convenience is improved.

A treatment tool control device 5049 controls driving of the energy treatment tool 5021 for cauterization and incision of tissue, sealing of a blood vessel, and the like. A pneumoperitoneum device 5051 sends a gas into the body cavity of the patient 5071 through the pneumoperitoneum tube 5019 to expand the body cavity for the purpose of securing a field of view by the endoscope 5001 and a work space for the operator. A recorder 5053 is a device that can record various types of information regarding the surgery. A printer 5055 is a device that can print the various types of information regarding the surgery in various formats such as a text, an image, or a graph.

Hereinafter, a particularly characteristic configuration in the endoscopic surgical system 5000 will be further described in detail.

(Support Arm Device)

The support arm device 5027 includes the base unit 5029 as a base and the arm unit 5031 extending from the base unit 5029. In the illustrated example, the arm unit 5031 includes the plurality of joint units 5033a, 5033b, and 5033c and the plurality of links 5035a and 5035b connected by the joint unit 5033b, but FIG. 1 illustrates the configuration of the arm unit 5031 in a simplified manner for simplification. In reality, the shapes, the number, and the arrangement of the joint units 5033a to 5033c and the links 5035a and 5035b, the directions of rotation axes of the joint units 5033a to 5033c, and the like can be appropriately set so that the arm unit 5031 has a desired degree of freedom. For example, the arm unit 5031 can be favorably configured to have six degrees of freedom or more. With the configuration, the endoscope 5001 can be freely moved within a movable range of the arm unit 5031. Therefore, the lens barrel 5003 of the endoscope 5001 can be inserted from a desired direction into the body cavity of the patient 5071.

Actuators are provided in the joint units 5033a to 5033c, and the joint units 5033a to 5033c are configured to be rotatable around a predetermined rotation axis by driving of the actuators. The driving of the actuators is controlled by the arm control device 5045, whereby rotation angles of the joint units 5033a to 5033c are controlled and driving of the arm unit 5031 is controlled. With the control, control of the position and posture of the endoscope 5001 can be realized. At this time, the arm control device 5045 can control the driving of the arm unit 5031 by various known control methods such as force control or position control.

For example, the driving of the arm unit 5031 may be appropriately controlled by the arm control device 5045 according to an operation input, and the position and posture of the endoscope 5001 may be controlled, by an appropriate operation input by the operator 5067 via the input device 5047 (including the foot switch 5057). With the control, the endoscope 5001 at the distal end of the arm unit 5031 can be moved from an arbitrary position to an arbitrary position, and then can be fixedly supported at the position after the movement. Note that the arm unit 5031 may be operated by a so-called master-slave system. In this case, the arm unit 5031 can be remotely operated by the user via the input device 5047 installed at a place distant from an operating room.

Furthermore, in a case where the force control is applied, the arm control device 5045 may perform so-called power assist control in which the arm control device 5045 receives an external force from the user and drives the actuators of the joint units 5033a to 5033c so that the arm unit 5031 is smoothly moved according to the external force. With the control, the user can move the arm unit 5031 with a relatively light force when moving the arm unit 5031 while being in direct contact with the arm unit 5031. Accordingly, the user can more intuitively move the endoscope 5001 with a simpler operation, and the user's convenience can be improved.

Here, in endoscopic surgery, the endoscope 5001 has been generally supported by a surgeon called scopist. In contrast, by use of the support arm device 5027, the position of the endoscope 5001 can be reliably fixed without manual operation, and thus an image of the operation site can be stably obtained and the surgery can be smoothly performed.

Note that the arm control device 5045 is not necessarily provided in the cart 5037. Furthermore, the arm control device 5045 is not necessarily one device. For example, the arm control device 5045 may be provided in each of the joint units 5033a to 5033c of the arm unit 5031 of the support arm device 5027, and the drive control of the arm unit 5031 may be realized by mutual cooperation of the plurality of arm control devices 5045.

(Light Source Device)

The light source device 5043 supplies irradiation light, which is used in capturing an operation site, to the endoscope 5001. The light source device 5043 includes, for example, an LED, a laser light source, or a white light source configured by a combination thereof. In a case where the white light source is configured by a combination of RGB laser light sources, output intensity and output timing of the respective colors (wavelengths) can be controlled with high accuracy. Therefore, white balance of a captured image can be adjusted in the light source device 5043. Further, in this case, the observation target is irradiated with the laser light from each of the RGB laser light sources in a time division manner, and the driving of the imaging element of the camera head 5005 is controlled in synchronization with the irradiation timing, so that images respectively corresponding to RGB can be captured in a time division manner. According to the method, a color image can be obtained without providing a color filter to the imaging element.

Furthermore, driving of the light source device 5043 may be controlled to change intensity of light to be output every predetermined time. The driving of the imaging element of the camera head 5005 is controlled in synchronization with change timing of the intensity of light, and images are acquired in a time division manner and are synthesized, whereby a high-dynamic range image without clipped blacks and flared highlights can be generated.

Further, the light source device 5043 may be configured to be able to supply light in a predetermined wavelength band corresponding to special light observation. In the special light observation, for example, so-called narrow band imaging is performed by radiating light in a narrower band than the irradiation light (in other words, white light) at the time of normal observation, using wavelength dependence of absorption of light in a body tissue, to capture a predetermined tissue such as a blood vessel in a mucosal surface layer at high contrast. Alternatively, in the special light observation, fluorescence observation to obtain an image by fluorescence generated by radiation of exciting light may be performed. In the fluorescence observation, irradiating the body tissue with exciting light to observe fluorescence from the body tissue (self-fluorescence observation), injecting a reagent such as indocyanine green (ICG) into the body tissue and irradiating the body tissue with exciting light corresponding to a fluorescence wavelength of the reagent to obtain a fluorescence image, or the like can be performed. The light source device 5043 can be configured to be able to supply narrow-band light and/or exciting light corresponding to such special light observation.

(Camera Head and CCU)

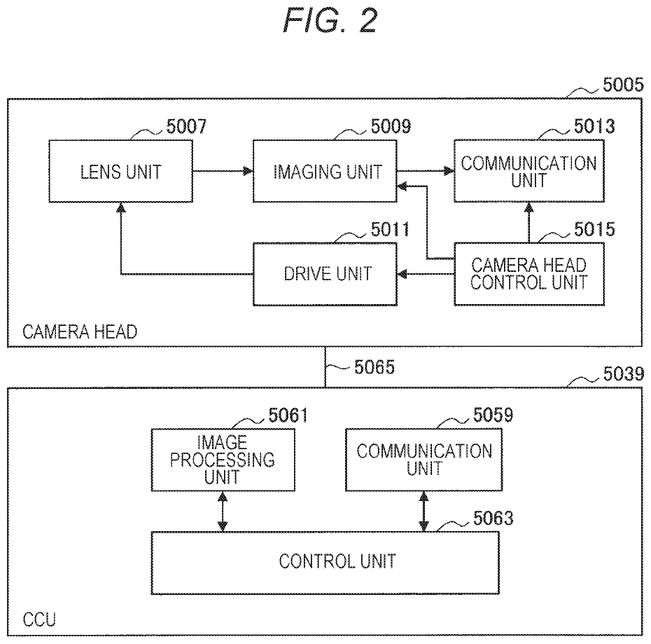

Functions of the camera head 5005 and the CCU 5039 of the endoscope 5001 will be described in more detail with reference to FIG. 2. FIG. 2 is a block diagram illustrating an example of functional configurations of the camera head 5005 and the CCU 5039 illustrated in FIG. 1.

Referring to FIG. 2, the camera head 5005 includes a lens unit 5007, an imaging unit 5009, a drive unit 5011, a communication unit 5013, and a camera head control unit 5015 as its functions. Furthermore, the CCU 5039 includes a communication unit 5059, an image processing unit 5061, and a control unit 5063 as its functions. The camera head 5005 and the CCU 5039 are communicatively connected with each other by a transmission cable 5065.

First, a functional configuration of the camera head 5005 will be described. The lens unit 5007 is an optical system provided in a connection portion between the lens unit 5007 and the lens barrel 5003. Observation light taken through the distal end of the lens barrel 5003 is guided to the camera head 5005 and enters the lens unit 5007. The lens unit 5007 is configured by a combination of a plurality of lenses including a zoom lens and a focus lens. Optical characteristics of the lens unit 5007 are adjusted to condense the observation light on a light receiving surface of an imaging element of the imaging unit 5009. Furthermore, the zoom lens and the focus lens are configured to have their positions on the optical axis movable for adjustment of the magnification and focal point of the captured image.

The imaging unit 5009 includes an imaging element, and is disposed at a rear stage of the lens unit 5007. The observation light having passed through the lens unit 5007 is focused on the light receiving surface of the imaging element, and an image signal corresponding to the observed image is generated by photoelectric conversion. The image signal generated by the imaging unit 5009 is provided to the communication unit 5013.

As the imaging element constituting the imaging unit 5009, for example, a complementary metal oxide semiconductor (CMOS)-type image sensor having Bayer arrangement and capable of color capturing is used. Note that, as the imaging element, for example, an imaging element that can capture a high-resolution image of 4K or more may be used. By obtainment of the image of the operation site with high resolution, the operator 5067 can grasp the state of the operation site in more detail and can more smoothly advance the surgery.

Furthermore, the imaging element constituting the imaging unit 5009 includes a pair of imaging elements for respectively obtaining image signals for right eye and for left eye corresponding to 3D display. With the 3D display, the operator 5067 can more accurately grasp the depth of biological tissue in the operation site. Note that, in a case where the imaging unit 5009 is configured as a multi-plate imaging unit, a plurality of systems of the lens units 5007 is provided corresponding to the imaging elements.

Furthermore, the imaging unit 5009 may not be necessarily provided in the camera head 5005. For example, the imaging unit 5009 may be provided immediately after the object lens inside the lens barrel 5003.

The drive unit 5011 includes an actuator, and moves the zoom lens and the focus lens of the lens unit 5007 by a predetermined distance along an optical axis by the control of the camera head control unit 5015. With the movement, the magnification and focal point of the captured image by the imaging unit 5009 can be appropriately adjusted.

The communication unit 5013 includes a communication device for transmitting or receiving various types of information to or from the CCU 5039. The communication unit 5013 transmits the image signal obtained from the imaging unit 5009 to the CCU 5039 through the transmission cable 5065 as raw data. At this time, to display the captured image of the operation site with low latency, the image signal is favorably transmitted by optical communication. This is because, in surgery, the operator 5067 performs surgery while observing the state of the affected part with the captured image, and thus display of a moving image of the operation site in as real time as possible is demanded for more safe and reliable surgery. In the case of the optical communication, a photoelectric conversion module that converts an electrical signal into an optical signal is provided in the communication unit 5013. The image signal is converted into the optical signal by the photoelectric conversion module, and is then transmitted to the CCU 5039 via the transmission cable 5065.

Furthermore, the communication unit 5013 receives a control signal for controlling driving of the camera head 5005 from the CCU 5039. The control signal includes information regarding the imaging conditions such as information for specifying a frame rate of the captured image, information for specifying an exposure value at the time of imaging, and/or information for specifying the magnification and the focal point of the captured image, for example. The communication unit 5013 provides the received control signal to the camera head control unit 5015. Note that the control signal from that CCU 5039 may also be transmitted by the optical communication. In this case, the communication unit 5013 is provided with a photoelectric conversion module that converts an optical signal into an electrical signal, and the control signal is converted into an electrical signal by the photoelectric conversion module and is then provided to the camera head control unit 5015.

Note that the imaging conditions such as the frame rate, exposure value, magnification, and focal point are automatically set by the control unit 5063 of the CCU 5039 on the basis of the acquired image signal. That is, a so-called auto exposure (AE) function, an auto focus (AF) function, and an auto white balance (AWB) function are incorporated in the endoscope 5001.

The camera head control unit 5015 controls the driving of the camera head 5005 on the basis of the control signal received from the CCU 5039 through the communication unit 5013. For example, the camera head control unit 5015 controls driving of the imaging element of the imaging unit 5009 on the basis of the information for specifying the frame rate of the captured image and/or the information for specifying exposure at the time of imaging. Furthermore, for example, the camera head control unit 5015 appropriately moves the zoom lens and the focus lens of the lens unit 5007 via the drive unit 5011 on the basis of the information for specifying the magnification and focal point of the captured image. The camera head control unit 5015 may further have a function to store information for identifying the lens barrel 5003 and the camera head 5005.

Note that the configuration of the lens unit 5007, the imaging unit 5009, and the like is arranged in a hermetically sealed structure having high airtightness and waterproofness, whereby the camera head 5005 can have resistance to autoclave sterilization processing.

Next, a functional configuration of the CCU 5039 will be described. The communication unit 5059 includes a communication device for transmitting or receiving various types of information to or from the camera head 5005. The communication unit 5059 receives the image signal transmitted from the camera head 5005 through the transmission cable 5065. At this time, as described above, the image signal can be favorably transmitted by the optical communication. In this case, the communication unit 5059 is provided with a photoelectric conversion module that converts an optical signal into an electrical signal, corresponding to the optical communication. The communication unit 5059 provides the image signal converted into the electrical signal to the image processing unit 5061.

Furthermore, the communication unit 5059 transmits a control signal for controlling driving of the camera head 5005 to the camera head 5005. The control signal may also be transmitted by the optical communication.

The image processing unit 5061 applies various types of image processing to the image signal as raw data transmitted from the camera head 5005. The image processing include various types of known signal processing such as development processing, high image quality processing (such as band enhancement processing, super resolution processing, noise reduction (NR) processing, and/or camera shake correction processing), and/or enlargement processing (electronic zoom processing), for example. Furthermore, the image processing unit 5061 performs wave detection processing for image signals for performing AE, AF, and AWB.

The image processing unit 5061 is configured by a processor such as a CPU or a GPU, and the processor is operated according to a predetermined program, whereby the above-described image processing and wave detection processing can be performed. Note that in a case where the image processing unit 5061 includes a plurality of GPUs, the image processing unit 5061 appropriately divides the information regarding the image signal and performs the image processing in parallel by the plurality of GPUs.

The control unit 5063 performs various types of control related to imaging of the operation site by the endoscope 5001 and display of the captured image. For example, the control unit 5063 generates a control signal for controlling driving of the camera head 5005. At this time, in a case where the imaging conditions are input by the user, the control unit 5063 generates the control signal on the basis of the input by the user. Alternatively, in a case where the AE function, the AF function, and the AWB function are incorporated in the endoscope 5001, the control unit 5063 appropriately calculates optimum exposure value, focal length, and white balance according to a result of the wave detection processing by the image processing unit 5061, and generates the control signal.

Furthermore, the control unit 5063 displays the image of the operation site on the display device 5041 on the basis of the image signal to which the image processing has been applied by the image processing unit 5061. At this time, the control unit 5063 recognizes various objects in the image of the operation site, using various image recognition technologies. For example, the control unit 5063 can recognize a surgical instrument such as forceps, a specific living body portion, blood, mist at the time of use of the energy treatment tool 5021, or the like, by detecting a shape of an edge, a color or the like of an object included in the operation site image. The control unit 5063 superimposes and displays various types of surgery support information on the image of the operation site, in displaying the image of the operation site on the display device 5041 using the result of recognition. The surgery support information is superimposed, displayed, and presented to the operator 5067, so that the surgery can be more safely and reliably advanced.

The transmission cable 5065 that connects the camera head 5005 and the CCU 5039 is an electrical signal cable supporting communication of electrical signals, an optical fiber supporting optical communication, or a composite cable thereof.

Here, in the illustrated example, the communication has been performed in a wired manner using the transmission cable 5065. However, the communication between the camera head 5005 and the CCU 5039 may be wirelessly performed. In a case where the communication between the camera head 5005 and the CCU 5039 is wirelessly performed, it is unnecessary to lay the transmission cable 5065 in the operating room. Therefore, the situation in which movement of medical staffs in the operating room is hindered by the transmission cable 5065 can be eliminated.

An example of an endoscopic surgical system 5000 to which the technology according to the present disclosure is applicable has been described. Note that, here, the endoscopic surgical system 5000 has been described as an example. However, a system to which the technology according to the present disclosure is applicable is not limited to this example. For example, the technique according to the present disclosure may be applied to a flexible endoscopic system for examination or a microsurgical system.

### 2. Specific Configuration Example of Support Arm Device

Next, a specific configuration example of a support arm device according to the embodiment of the present disclosure will be described in detail. The support arm device described below is an example configured as a support arm device that supports an endoscope at a distal end of an arm unit. However, the present embodiment is not limited to the example.

Furthermore, in a case where the support arm device according to the embodiment of the present disclosure is applied to the medical field, the support arm device according to the embodiment of the present disclosure can function as a medical support arm device.

<2-1. Appearance of Support Arm Device>

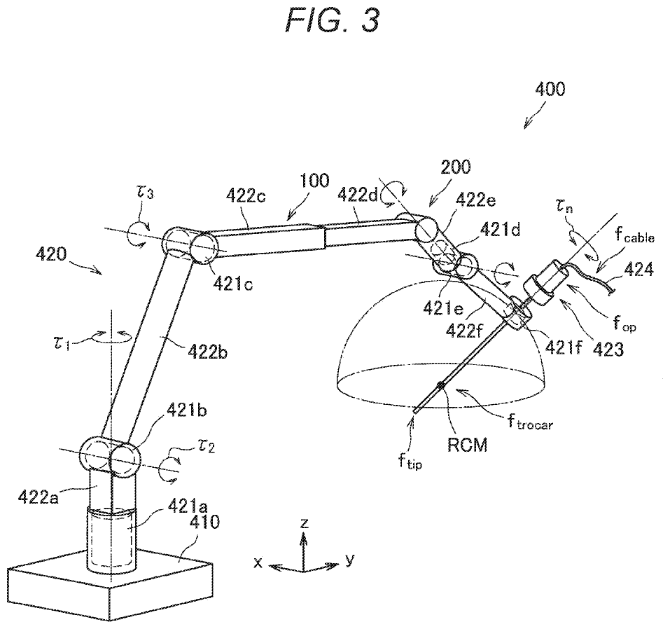

First, a schematic configuration of a support arm device 400 according to the present embodiment will be described with reference to FIG. 3. FIG. 3 is a schematic view illustrating an appearance of the support arm device 400 according to the present embodiment.

The support arm device 400 according to the present embodiment includes a base unit 410 and an arm unit 420. The base unit 410 is a base of the support arm device 400, and the arm unit 420 is extended from the base unit 410. Furthermore, although not illustrated in FIG. 3, a control unit that integrally controls the support arm device 400 may be provided in the base unit 410, and driving of the arm unit 420 may be controlled by the control unit. The control unit includes various signal processing circuits, such as a CPU and a DSP, for example.

The arm unit 420 includes a plurality of active joint units 421a to 421f, a plurality of links 422a to 422f, and an endoscope device 423 as a distal end unit provided at a distal end of the arm unit 420.

The links 422a to 422f are substantially rod-like members. One end of the link 422a is connected to the base unit 410 via the active joint unit 421a, the other end of the link 422a is connected to one end of the link 422b via the active joint unit 421b, and the other end of the link 422b is connected to one end of the link 422c via the active joint unit 421c. The other end of the link 422c is connected to the link 422d via a passive slide mechanism 100, and the other end of the link 422d is connected to one end of the link 422e via a passive joint unit 200. The other end of the link 422e is connected to one end of the link 422f via the active joint units 421d and 421e. The endoscope device 423 is connected to the distal end of the arm unit 420, in other words, the other end of the link 422f, via the active joint unit 421f. The respective ends of the plurality of links 422a to 422f are connected one another by the active joint units 421a to 421f, the passive slide mechanism 100, and the passive joint unit 200 with the base unit 410 as a fulcrum, as described above, so that an arm shape extended from the base unit 410 is configured.

Actuators provided in the respective active joint units 421a to 421f of the arm unit 420 are driven and controlled, so that the position and posture of the endoscope device 423 are controlled. In the present embodiment, the endoscope device 423 has a distal end enter a body cavity of a patient, which is an operation site, and captures a partial region of the operation site. Note that the distal end unit provided at the distal end of the arm unit 420 is not limited to the endoscope device 423, and various medical instruments may be connected to the distal end of the arm unit 420 as the distal end units. Thus, the support arm device 400 according to the present embodiment is configured as a medical support arm device provided with a medical instrument.

Here, hereinafter, the support arm device 400 will be described by defining coordinate axes as illustrated in FIG. 3. Furthermore, an up-down direction, a front-back direction, and a right-left direction will be defined in accordance with the coordinate axes. In other words, the up-down direction with respect to the base unit 410 installed on a floor is defined as a z-axis direction and the up-down direction Furthermore, a direction orthogonal to the z axis and in which the arm unit 420 is extended from the base unit 410 (in other words, a direction in which the endoscope device 423 is located with respect to the base unit 410) is defined as a y-axis direction and the front-back direction. Moreover, a direction orthogonal to the y axis and the z axis is defined as an x-axis direction and the right-left direction.

The active joint units 421a to 421f rotatably connect the links to one another. The active joint units 421a to 421f include actuators, and have a rotation mechanism that is rotationally driven about a predetermined rotation axis by driving of the actuators. By controlling rotational driving of each of the active joint units 421a to 421f, driving of the arm unit 420 such as extending or contracting (folding) of the arm unit 420 can be controlled. Here, the driving of the active joint units 421a to 421f can be controlled by, for example, known whole body coordination control and ideal joint control. As described above, since the active joint units 421a to 421f have the rotation mechanism, in the following description, the drive control of the active joint units 421a to 421f specifically means control of rotation angles and/or generated torque (torque generated by the active joint units 421a to 421f) of the active joint units 421a to 421f.

The passive slide mechanism 100 is an aspect of a passive form change mechanism, and connects the link 422c and the link 422d to be able to move forward and backward along a predetermined direction. For example, the passive slide mechanism 100 may connect the link 422c and the link 422d in a linearly movable manner. However, the forward/backward motion of the link 422c and the link 422d is not limited to the linear motion, and may be forward/backward motion in a direction of forming an arc. The passive slide mechanism 100 is operated in the forward/backward motion by a user, for example, and makes a distance between the active joint unit 421c on the one end side of the link 422c and the passive joint unit 200 variable. Thereby, the entire form of the arm unit 420 can change.

The passive joint unit 200 is one aspect of the passive form change mechanism, and rotatably connects the link 422d and the link 422e to each other. The passive joint unit 200 is rotatably operated by the user, for example, and makes an angle made by the link 422d and the link 422e variable. Thereby, the entire form of the arm unit 420 can change.

Note that, in the present specification, a “posture of the arm unit” refers to a state of the arm unit changeable by the drive control of the actuators provided in the active joint units 421a to 421f by the control unit in a state where the distance between active joint units adjacent across one or a plurality of links is constant. Furthermore, a “form of the arm unit” refers to a state of the arm unit changeable as the distance between active joint units adjacent across a link or an angle between links connecting adjacent active joint units changes with the operation of the passive form change mechanism.

The support arm device 400 according to the present embodiment includes the six active joint units 421a to 421f and realizes six degrees of freedom with respect to the driving of the arm unit 420. That is, while the drive control of the support arm device 400 is realized by the drive control of the six active joint units 421a to 421f by the control unit, the passive slide mechanism 100 and the passive joint unit 200 are not the targets of the drive control by the control unit.

Specifically, as illustrated in FIG. 3, the active joint units 421a, 421d, and 421f are provided to have long axis directions of the connected links 422a and 422e and a capture direction of the connected endoscope device 423 as rotation axis directions. The active joint units 421b, 421c, and 421e are provided to have the x-axis direction that is a direction in which connection angles of the connected links 422a to 422c, 422e, and 422f and the connected endoscope device 423 are changed in a y-z plane (a plane defined by the y axis and the z axis) as rotation axis directions. As described above, in the present embodiment, the active joint units 421a, 421d, and 421f have a function to perform so-called yawing, and the active joint units 421b, 421c, and 421e have a function to perform so-called pitching.

With the above configuration of the arm unit 420, the support arm device 400 according to the present embodiment realizes the six degrees of freedom with respect to the driving of the arm unit 420, whereby freely moving the endoscope device 423 within the movable range of the arm unit 420. FIG. 3 illustrates a hemisphere as an example of a movable range of the endoscope device 423. In a case where a central point RCM (remote motion center) of the hemisphere is a capture center of the operation site captured by the endoscope device 423, the operation site can be captured from various angles by moving the endoscope device 423 on a spherical surface of the hemisphere in a state where the capture center of the endoscope device 423 is fixed to the central point of the hemisphere.

The schematic configuration of the support arm device 400 according to the present embodiment has been described above. Next, the whole body coordination control and the ideal joint control for controlling the driving of the arm unit 420, in other words, the driving of the joint units 421a to 421f in the support arm device 400 according to the present embodiment will be described.

<2-2. Generalized Inverse Dynamics>

Next, an overview of generalized inverse dynamics used for the whole body coordination control of the support arm device 400 in the present embodiment will be described.

The generalized inverse dynamics is basic operation in the whole body coordination control of a multilink structure configured by connecting a plurality of links by a plurality of joint units (for example, the arm unit 420 illustrated in FIG. 2 in the present embodiment), for converting motion purposes regarding various dimensions in various operation spaces into torque to be caused in the plurality of joint units in consideration of various constraint conditions.

The operation space is an important concept in force control of a robot device. The operation space is a space for describing a relationship between force acting on the multilink structure and acceleration of the multilink structure. When the drive control of the multilink structure is performed not by position control but by force control, the concept of the operation space is required in a case of using a contact between the multilink structure and an environment as a constraint condition. The operation space is, for example, a joint space, a Cartesian space, a momentum space, or the like, which is a space to which the multilink structure belongs.

The motion purpose represents a target value in the drive control of the multilink structure, and is, for example, a target value of a position, a speed, an acceleration, a force, an impedance, or the like of the multilink structure to be achieved by the drive control.

The constraint condition is a constraint condition regarding the position, speed, acceleration, force, or the like of the multilink structure, which is determined according to a shape or a structure of the multilink structure, an environment around the multilink structure, settings by the user, and the like. For example, the constraint condition includes information regarding a generated force, a priority, presence/absence of a non-drive joint, a vertical reaction force, a friction weight, a support polygon, and the like.

In the generalized dynamics, to establish both stability of numerical calculation and real time processing efficiency, an arithmetic algorithm includes a virtual force determination process (virtual force calculation processing) as a first stage and a real force conversion process (real force calculation processing) as a second stage. In the virtual force calculation processing as the first stage, a virtual force that is a virtual force required for achievement of each motion purpose and acting on the operation space is determined while considering the priority of the motion purpose and a maximum value of the virtual force. In the real force calculation processing as the second stage, the above-obtained virtual force is converted into a real force realizable in the actual configuration of the multilink structure, such as a joint force or an external force, while considering the constraints regarding the non-drive joint, the vertical reaction force, the friction weight, the support polygon, and the like. Hereinafter, the virtual force calculation processing and the real force calculation processing will be described in detail. Note that, in the description of the virtual force calculation processing and the real force calculation processing below and the real force calculation processing to be described below, description may be performed using the configuration of the arm unit 420 of the support arm device 400 according to the present embodiment illustrated in FIG. 3 as a specific example, in order to facilitate understanding.

(2-2-1. Virtual Force Calculation Processing)

A vector configured by a certain physical quantity at each joint unit of the multilink structure is called generalized variable q (also referred to as a joint value q or a joint space q). An operation space x is defined by the following expression (1) using a time derivative value of the generalized variable q and the Jacobian J.

[Math. 1]

\dot{x}=J\dot{q} (1)

In the present embodiment, for example, q is a rotation angle of the joint units 421a to 421f of the arm unit 420. An equation of motion regarding the operation space x is described by the following expression (2).

[Math. 2]

\ddot{x}=Λ⁻¹f+c (2)

Here, f represents a force acting on the operation space x. Furthermore, Λ⁻¹ is an operation space inertia inverse matrix, and c is called operation space bias acceleration, which are respectively expressed by the following expressions (3) and (4).

[Math. 3]

Λ⁻¹=JH⁻¹J^{T} (3)

c=JH⁻¹(τ−b)+\dot{J}\dot{q} (4)

Note that H represents a joint space inertia matrix, τ represents a joint force corresponding to the joint value q (for example, the generated torque at the joint units 421a to 421f), and b represents gravity, a Coriolis force, and a centrifugal force.

In the generalized inverse dynamics, it is known that the motion purpose of the position and speed regarding the operation space x can be expressed as an acceleration of the operation space x. At this time, the virtual force fᵥ to act on the operation space x to realize an operation space acceleration that is a target value given as the motion purpose can be obtained by solving a kind of linear complementary problem (LCP) as in the expression (5) below according to the above expression (1).

$$\begin{matrix}[ Math . 4 ] & \\ w + \ddot{x} = {Λ}^{- 1} {f}_{v} + c \\ s . t . \{ \begin{matrix}( ( {w}_{i} < 0 ) ⋂ ( {f}_{{v}_{i}} = {U}_{i} ) ) ⋃ \\ ( ( {w}_{i} > 0 ) ⋂ ( {f}_{{v}_{i}} = {L}_{i} ) ) ⋃ \\ ( ( {w}_{i} = 0 ) ⋂ ( {L}_{i} < {f}_{{v}_{i}} < {U}_{i} ) )\end{matrix} & ( 5 )\end{matrix}$$

Here, Lᵢ and Uᵢ respectively represent a negative lower limit value (including −∞) of an i-th component of fᵥ and a positive upper limit value (including +∞) of the i-th component of fᵥ. The above LCP can be solved using, for example, an iterative method, a pivot method, a method applying robust acceleration control, or the like.

Note that the operation space inertia inverse matrix Λ⁻¹ and the bias acceleration c have a large calculation cost when calculated according to the expressions (3) and (4) that are defining expressions. Therefore, a method of calculating the processing of calculating the operation space inertia inverse matrix Λ⁻¹ at a high speed by applying a quasi-dynamics operation (FWD) for obtaining a generalized acceleration (joint acceleration) from the generalized force (joint force τ) of the multilink structure has been proposed. Specifically, the operation space inertia inverse matrix Λ⁻¹ and the bias acceleration c can be obtained from information regarding forces acting on the multilink structure (for example, the joint units 421a to 421f of the arm unit 420), such as the joint space q, the joint force τ, and the gravity g by using the forward dynamics operation FWD. The operation space inertia inverse matrix Λ⁻¹ can be calculated with a calculation amount of O (N) with respect to the number (N) of the joint units by applying the forward dynamics operation FWD regarding the operation space.

Here, as a setting example of the motion purpose, a condition for achieving the target value (expressed by adding a superscript bar to second-order differentiation of x) of the operation space acceleration with a virtual force fᵥᵢ equal to or smaller than an absolute value Fᵢ can be expressed by the following expression (6).

[Math. 5]

Lᵢ=−Fᵢ,

Uᵢ=Fᵢ,

\ddot{x}ᵢ=\ddot{x̅}ᵢ (6)

Furthermore, as described above, the motion purpose regarding the position and speed of the operation space x can be expressed as the target value of the operation space acceleration, and is specifically expressed by the following expression (7) (the target value of the position and speed of the operation space x is expressed by x and adding the superscript bar to first-order differentiation of x).

[Math. 6]

\ddot{x̅}ᵢ=Kₚ(x̅ᵢ−xᵢ)+Kᵥ(\ddot{x̅}ᵢ−\dot{x}ᵢ) (7)

In addition, by use of a concept of decomposition operation space, the motion purpose regarding an operation space (momentum, Cartesian relative coordinates, interlocking joint, or the like) expressed by a linear sum of other operation spaces. Note that it is necessary to give priority to competing motion purposes. The above LCP can be solved for each priority in ascending order from a low priority, and the virtual force obtained by the LCP in the previous stage can be made to act as a known external force of the LCP in the next stage.

(2-2-2. Real Force Calculation Processing)

In the real force calculation processing as the second stage of the generalized inverse dynamics, processing of replacing the virtual force fᵥ obtained in the above (2-2-1. Virtual Force Determination Process) with real joint force and external force is performed. A condition for realizing the generalized force τᵥ=Jᵥ^{T}fᵥ by the virtual force with a generated torque τₐ and an external force fₑ generated in the joint unit is expressed by the following expression (8).

$$\begin{matrix}[ Math . 7 ] & \\ [ \begin{matrix}{J}_{vu}^{T} \\ {J}_{va}^{T}\end{matrix} ] ( {f}_{v} - Δ {f}_{v} ) = [ \begin{matrix}{J}_{eu}^{T} \\ {J}_{ea}^{T}\end{matrix} ] {f}_{e} + [ \begin{matrix}0 \\ {τ}_{a}\end{matrix} ] & ( 8 )\end{matrix}$$

Here, the suffix a represents a set of drive joint units (drive joint set), and the suffix u represents a set of non-drive joint units (non-drive joint set). In other words, the upper part of the above expression (8) represents balance of the forces of the space (non-drive joint space) by the non-drive joint units, and the lower part represents balance of the forces of the space (drive joint space) by the drive joint units. Jᵥᵤ and Jᵥₐ are respectively a non-drive joint component and a drive joint component of the Jacobian regarding the operation space where the virtual force fᵥ acts. Jₑᵤ and Jₑₐ are a non-drive joint component and a drive joint component of the Jacobian regarding the operation space where the external force fₑ acts. Δfᵥ represents an unrealizable component with the real force, of the virtual force fᵥ.

The upper part of the expression (8) is undefined. For example, fₑ and Δfᵥ can be obtained by solving a quadratic programing problem (QP) as described in the following expression (9).

$$\begin{matrix}[ Math . 8 ] & \\ min \frac{1}{2} {ɛ}^{T} {Q}_{1} ɛ + \frac{1}{2} {ξ}^{T} {Q}_{2} ξ \\ s . t . U ξ \geq v & ( 9 )\end{matrix}$$

Here, ε is a difference between both sides of the upper part of the expression (8), and represents an equation error of the expression (8). ξ is a connected vector of fₑ and Δfᵥ and represents a variable vector. Q₁ and Q₂ are positive definite symmetric matrices that represent weights at minimization. Furthermore, inequality constraint of the expression (9) is used to express the constraint condition regarding the external force such as the vertical reaction force, friction cone, maximum value of the external force, or support polygon. For example, the inequality constraint regarding a rectangular support polygon is expressed by the following expression (10).

[Math. 9]

|Fₓ|≤μₜF_{z},

|F_{y}|≤μₜF_{z},

F_{z}≥0,

|Mₓ|≤dₜF_{z},

|M_{y}|≤dₓF_{z},

|Mₓ|≤μᵣF_{z}, (10)

Here, z represents a normal direction of a contact surface, and x and y represent orthogonal two-tangent directions perpendicular to z. (Fₓ, F_{y}, F_{z}) and (Mₓ, M_{y}, M_{z}) represent an external force and an external force moment acting on a contact point. μₜ and μᵣ are friction coefficients regarding translation and rotation, respectively. (dₓ, d_{y}) represents the size of the support polygon.

From the above expressions (9) and (10), solutions fₑ and Δfᵥ of a minimum norm or a minimum error are obtained. By substituting fₑ and Δfᵥ obtained from the above expression (9) into the lower part of the above expression (8), the joint force τₐ necessary for realizing the motion purpose can be obtained.

In a case of a system where a base is fixed and there is no non-drive joint, all virtual forces can be replaced only with the joint force, and fₑ=0 and Δfᵥ=0 can be set in the above expression (8). In this case, the following expression (11) can be obtained for the joint force τₐ from the lower part of the above expression (8).

[Math. 10]

τₐ=Jᵥₐ^{T}fᵥ (11)

The whole body coordination control using the generalized inverse dynamics according to the present embodiment has been described. By sequentially performing the virtual force calculation processing and the real force calculation processing as described above, the joint force τₐ for achieving a desired motion purpose can be obtained. In other words, conversely speaking, by reflecting the calculated joint force τₐ in a theoretical model in the motion of the joint units 421a to 421f, the joint units 421a to 421f are driven to achieve the desired motion purpose.

Note that, regarding the whole body coordination control using the generalized inverse dynamics described so far, in particular, details of the process of deriving the virtual force fᵥ, the method of solving the LCP to obtain the virtual force fᵥ, the solution of the QP problem, and the like, reference can be made to Japanese Patent Application Laid-Open Nos. 2009-95959 and 2010-188471, which are prior patent applications filed by the present applicant, for example.

<2-3. Ideal Joint Control>

Next, the ideal joint control according to the present embodiment will be described. The motion of each of the joint units 421a to 421f is modeled by the equation of motion of the second-order lag system of the following expression (12).

[Math. 11]

Iₐ\ddot{q}=τₐ+τₑ−vₐ\dot{q} (12)

Here, Iₐ represents moment of inertia (inertia) at the joint unit, τₐ represents the generated torque of the joint units 421a to 421f, τₑ represents external torque acting on each of the joint units 421a to 421f from the outside, and νₑ represents a viscous drag coefficient in each of the joint units 421a to 421f. The above expression (12) can also be said to be a theoretical model that represents the motion of the actuators in the joint units 421a to 421f.

τₐ that is the real force to act on each of the joint units 421a to 421f for realizing the motion purpose can be calculated using the motion purpose and the constraint condition by the operation using the generalized inverse dynamics described in <2-2. Generalized Inverse Dynamics> above. Therefore, ideally, by applying each calculated τₐ to the above expression (12), a response according to the theoretical model illustrated in the above expression (12) is realized, in other words, the desired motion purpose should be achieved.

However, in practice, errors (modeling errors) may occur between the motions of the joint units 421a to 421f and the theoretical model as illustrated in the above expression (12), due to the influence of various types of disturbance. The modeling errors can be roughly classified into those due to mass property such as weight, center of gravity, inertia tensor of the multilink structure, and those due to friction, inertia, and the like inside joint units 421a to 421f. Among them, the modeling errors due to the former mass property can be relatively easily reduced at the time of constructing the theoretical model by improving the accuracy of computer aided design (CAD) data and applying an identification method.

Meanwhile, the modeling errors due to the latter friction, inertia, and the like inside the joint units 421a to 421f are caused by phenomena that are difficult to model, such as friction in a reduction gear 426 of the joint units 421a to 421f, for example, and a modeling error that cannot be ignored may remain during model construction. Furthermore, there is a possibility that an error occurs between the values of the inertia Iₐ and the viscous drag coefficient νₑ in the above expression (12) and the values in the actual joint units 421a to 421f. These errors that are difficult to model can become the disturbance in the drive control of the joint units 421a to 421f. Therefore, in practice, the motions of the joint units 421a to 421f may not respond according to the theoretical model illustrated in the above expression (12), due to the influence of such disturbance. Therefore, even when the real force τₐ, which is a joint force calculated by the generalized inverse dynamics, is applied, there may be a case where the motion purpose that is the control target is not achieved. In the present embodiment, correcting the responses of the joint units 421a to 421f so as to perform ideal responses according to the theoretical model illustrated in the above expression (12), by adding an active control system to each of the joint units 421a to 421f, is considered. Specifically, in the present embodiment, not only performing friction compensation type torque control using the torque sensors 428 and 428a of the joint units 421a to 421f but also performing an ideal response according to the theoretical values up to the inertia Iₐ and the viscous drag coefficient νₐ to the required generated torque τₐ and external torque τₑ.

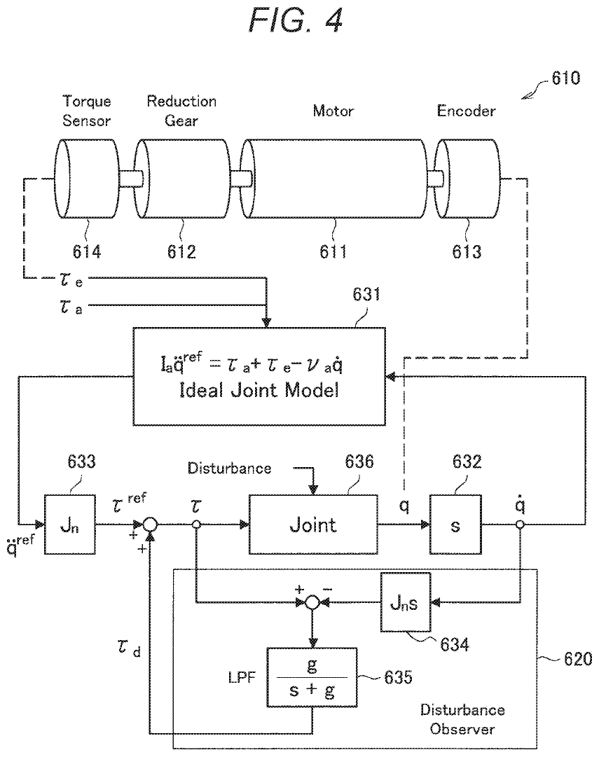

In the present embodiment, control of the driving of the joint units 421a to 421f of the support arm device 400 to perform ideal responses as described in the above expression (12) is called ideal joint control. Here, in the following description, an actuator controlled to be driven by the ideal joint control is also referred to as a virtualized actuator (VA) because of performing an ideal response. Hereinafter, the ideal joint control according to the present embodiment will be described with reference to FIG. 4.

FIG. 4 is an explanatory diagram for describing the ideal joint control according to an embodiment of the present disclosure. Note that FIG. 4 schematically illustrates a conceptual arithmetic unit that performs various operations regarding the ideal joint control in blocks.

Here, a response of an actuator 610 according to the theoretical model expressed by the above expression (12) is nothing less than achievement of the rotation angular acceleration on the left side when the right side of the expression (12) is given. Furthermore, as illustrated in the above expression (12), the theoretical model includes an external torque term τₑ acting on the actuator 610. In the present embodiment, the external torque term τₑ is measured by a torque sensor 614 in order to perform the ideal joint control. Furthermore, a disturbance observer 620 is applied to calculate a disturbance estimation value I_{d} that is an estimation value of a torque due to disturbance on the basis of a rotation angle q of the actuator 610 measured by an encoder 613.

A block 631 represents an arithmetic unit that performs an operation according to an ideal joint model of the joint units 421a to 421f illustrated in the above expression (12). The block 631 can output a rotation angular acceleration target value (a second derivative of a rotation angle target value q^{ref} described on the left side of the above expression (12), using the generated torque τₐ, the external torque τₑ, and the rotation angular speed (first-order differentiation of the rotation angle q) as inputs.

In the present embodiment, the generated torque τₐ calculated by the method described in <2-2. Generalized inverse dynamics> above and the external torque τₑ measured by the torque sensor 614 are input to the block 631. Meanwhile, when the rotation angle q measured by the encoder 613 is input to a block 632 representing an arithmetic unit that performs a differential operation, the rotation angular speed (the first-order differentiation of the rotation angle q) is calculated. When the rotation angular speed calculated in the block 632 is input to the block 631 in addition to the generated torque τₐ and the external torque τₑᵣ the rotation angular acceleration target value is calculated by the block 631. The calculated rotation angular acceleration target value is input to a block 633.

The block 633 represents an arithmetic unit that calculates a torque generated in the actuator 610 on the basis of the rotation angular acceleration of the actuator 610. In the present embodiment, specifically, the block 633 can obtain a torque target value τ^{ref} by multiplying the rotation angular acceleration target value by nominal inertia Jₙ in the actuator 610. In the ideal response, the desired motion purpose should be achieved by causing the actuator 610 to generate the torque target value τ^{ref}. However, as described above, there is a case where the influence of the disturbance or the like occurs in the actual response. Therefore, in the present embodiment, the disturbance observer 620 calculates the disturbance estimation value τ_{d} and corrects the torque target value τ^{ref} using the disturbance estimation value τ_{d}.

A configuration of the disturbance observer 620 will be described. As illustrated in FIG. 4, the disturbance observer 620 calculates the disturbance estimation value τ_{d} on the basis of the torque command value T and the rotation angular speed output from the rotation angle q measured by the encoder 613. Here, the torque command value τ is a torque value to be finally generated in the actuator 610 after the influence of disturbance is corrected. For example, win a case where the disturbance estimation value τ_{d} is not calculated, the torque command value τ becomes the torque target value τ^{ref}.

The disturbance observer 620 includes a block 634 and a block 635. The block 634 represents an arithmetic unit that calculates a torque generated in the actuator 610 on the basis of the rotation angular speed of the actuator 610. In the present embodiment, specifically, the rotation angular speed calculated by the block 632 from the rotation angle q measured by the encoder 613 is input to the block 634. The block 634 obtains the rotation angular acceleration by performing an operation represented by a transfer function Jₙs, in other words, by differentiating the rotation angular speed, and further multiplies the calculated rotation angular acceleration by the nominal inertia Jₙ, thereby calculating an estimation value of the torque actually acting on the actuator 610 (torque estimation value).

In the disturbance observer 620, the difference between the torque estimation value and the torque command value τ is obtained, whereby the disturbance estimation value τ_{d}, which is the value of the torque due to the disturbance, is estimated. Specifically, the disturbance estimation value τ_{d} may be a difference between the torque command value τ in the control of the preceding cycle and the torque estimation value in the current control. Since the torque estimation value calculated by the block 634 is based on the actual measurement value and the torque command value τ calculated by the block 633 is based on the ideal theoretical model of the joint units 421a to 421f illustrated in the block 631, the influence of the disturbance, which is not considered in the theoretical model, can be estimated by taking the difference between the torque estimation value and the torque command value τ.

Furthermore, the disturbance observer 620 is provided with a low pass filter (LPF) illustrated in a block 635 to prevent system divergence. The block 635 outputs only a low frequency component to the input value by performing an operation represented by a transfer function g/(s+g) to stabilize the system. In the present embodiment, the difference value between the torque estimation value and the torque command value τ^{ref} calculated by the block 634 is input to the block 635, and a low frequency component of the difference value is calculated as the disturbance estimation value τ_{d}.

In the present embodiment, feedforward control to add the disturbance estimation value τ_{d} calculated by the disturbance observer 620 to the torque target value τ^{ref} is performed, whereby the torque command value T that is the torque value to be finally generated in the actuator 610 is calculated. Then, the actuator 610 is driven on the basis of the torque command value τ. Specifically, the torque command value τ is converted into a corresponding current value (current command value), and the current command value is applied to a motor 611, so that the actuator 610 is driven.

As described above, with the configuration described with reference to FIG. 4, the response of the actuator 610 can be made to follow the target value even in a case where there is a disturbance component such as friction in the drive control of the joint units 421a to 421f according to the present embodiment. Furthermore, with regard to the drive control of the joint units 421a to 421f, an ideal response according to the inertia Iₐ and the viscous drag coefficient νₐ assumed by the theoretical model can be made.

Note that, for details of the above-described ideal joint control, Japanese Patent Application Laid-Open No. 2009-269102, which is a prior patent application filed by the present applicant, can be referred to, for example.

The generalized inverse dynamics used in the present embodiment has been described, and the ideal joint control according to the present embodiment has been described with reference to FIG. 4. As described above, in the present embodiment, the whole body coordination control, in which the drive parameters of the joint units 421a to 421f (for example, the generated torque values of the joint units 421a to 421f) for achieving the motion purpose of the arm unit 420 are calculated in consideration of the constraint condition, is performed using the generalized inverse dynamics. Furthermore, as described with reference to FIG. 4, in the present embodiment, the ideal joint control that realizes the ideal response based on the theoretical model in the drive control of the joint units 421a to 421f by performing correction of the generated torque value, which has been calculated in the whole body coordination control using the generalized inverse dynamics, in consideration of the influence of the disturbance, is performed. Therefore, in the present embodiment, highly accurate drive control that achieves the motion purpose becomes possible with regard to the driving of the arm unit 420.

<2-4. Configuration of Arm Control System>

Next, a configuration of an arm control system according to the present embodiment, in which the whole body coordination control and the ideal joint control described in <2-2. Generalized Inverse Dynamics> and <2-3. Ideal Joint Control> above are applied to drive control of an arm device will be described.

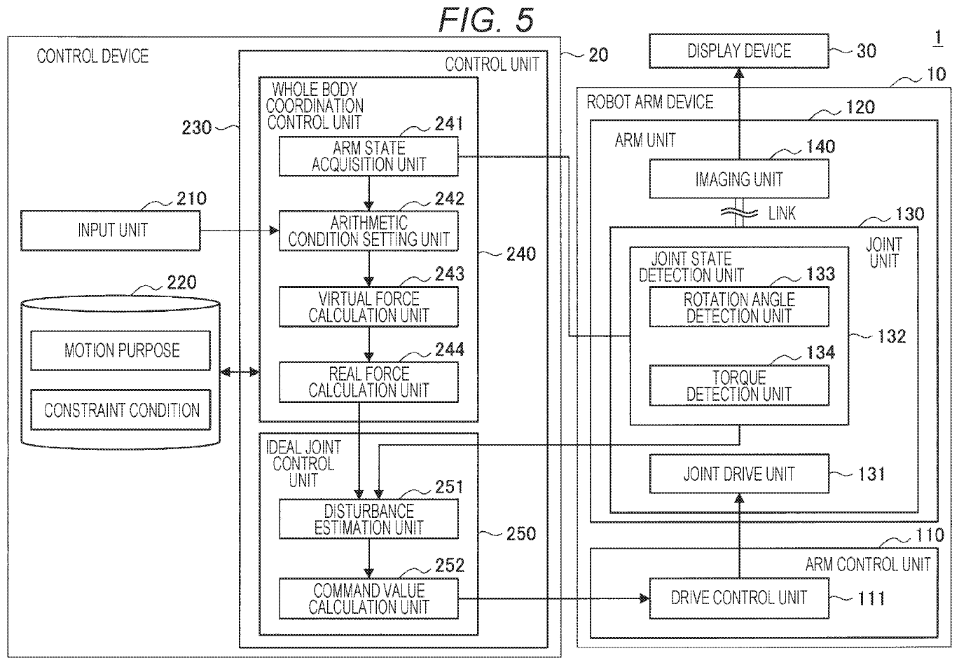

A configuration example of an arm control system according to an embodiment of the present disclosure will be described with reference to FIG. 5. FIG. 5 is a functional block diagram illustrating a configuration example of an arm control system according to an embodiment of the present disclosure. Note that, in the arm control system illustrated in FIG. 5, a configuration related to drive control of an arm unit of an arm device will be mainly illustrated.

Referring to FIG. 5, an arm control system 1 according to an embodiment of the present disclosure includes an arm device 10, a control device 20, and a display device 30. In the present embodiment, the control device 20 performs various operations in the whole body coordination control described in <2-2. Generalized Inverse Dynamics> and the ideal joint control described in <2-3. Ideal Joint Control> above, and driving of the arm unit of the arm device 10 is controlled on the basis of an operation result. Furthermore, the arm unit of the arm device 10 is provided with an imaging unit 140 described below, and an image captured by the imaging unit 140 is displayed on a display screen of the display device 30. Hereinafter, configurations of the arm device 10, the control device 20, and the display device 30 will be described in detail.

The arm device 10 includes the arm unit that is a multilink structure including a plurality of joint units and a plurality of links, and drives the arm unit within a movable range to control the position and posture of a distal end unit provided at a distal end of the arm unit. The arm device 10 corresponds to the support arm device 400 illustrated in FIG. 3.

Referring to FIG. 5, the arm device 10 includes an arm control unit 110 and an arm unit 120. Furthermore, the arm unit 120 includes a joint unit 130 and the imaging unit 140.

The arm control unit 110 integrally controls the arm device 10 and controls driving of the arm unit 120. The arm control unit 110 corresponds to the control unit (not illustrated in FIG. 3) described with reference to FIG. 3. Specifically, the arm control unit 110 includes a drive control unit 111. Driving of the joint unit 130 is controlled by the control of the drive control unit 111, so that the driving of the arm unit 120 is controlled. More specifically, the drive control unit 111 controls a current amount to be supplied to a motor in an actuator of the joint unit 130 to control the number of rotations of the motor, thereby controlling a rotation angle and generated torque in the joint unit 130. However, as described above, the drive control of the arm unit 120 by the drive control unit 111 is performed on the basis of the operation result in the control device 20. Therefore, the current amount to be supplied to the motor in the actuator of the joint unit 130, which is controlled by the drive control unit 111, is a current amount determined on the basis of the operation result in the control device 20.

The arm unit 120 is a multilink structure including a plurality of joints and a plurality of links, and driving of the arm unit 120 is controlled by the control of the arm control unit 110. The arm unit 120 corresponds to the arm unit 420 illustrated in FIG. 3. The arm unit 120 includes the joint unit 130 and the imaging unit 140. Note that, since functions and structures of the plurality of joint units included in the arm unit 120 are similar to one another, FIG. 5 illustrates a configuration of one joint unit 130 as a representative of the plurality of joint units.

The joint unit 130 rotatably connects the links with each other in the arm unit 120, and drives the arm unit 120 as rotational driving of the joint unit 130 is controlled by the control of the arm control unit 110. The joint unit 130 corresponds to the joint units 421a to 421f illustrated in FIG. 3. Furthermore, the joint unit 130 includes an actuator.

The joint unit 130 includes a joint drive unit 131 and a joint state detection unit 132.

The joint drive unit 131 is a drive mechanism in the actuator of the joint unit 130, and the joint unit 130 is rotationally driven as the joint drive unit 131 is driven. The driving of the joint drive unit 131 is controlled by the drive control unit 111. For example, the joint drive unit 131 is a configuration corresponding to the motor and a motor driver, and the joint drive unit 131 being driven corresponds to the motor driver driving the motor with the current amount according to a command from the drive control unit 111.

The joint state detection unit 132 detects a state of the joint unit 130. Here, the state of the joint unit 130 may mean a state of motion of the joint unit 130. For example, the state of the joint unit 130 includes information of the rotation angle, rotation angular speed, rotation angular acceleration, generated torque of the joint unit 130, and the like. In the present embodiment, the joint state detection unit 132 has a rotation angle detection unit 133 that detects the rotation angle of the joint unit 130 and a torque detection unit 134 that detects the generated torque and the external torque of the joint unit 130. Note that the rotation angle detection unit 133 and the torque detection unit 134 correspond to an encoder and a torque sensor of the actuator, respectively. The joint state detection unit 132 transmits the detected state of the joint unit 130 to the control device 20.

The imaging unit 140 is an example of the distal end unit provided at the distal end of the arm unit 120, and acquires an image of a capture target. The imaging unit 140 corresponds to the imaging unit 423 illustrated in FIG. 3. Specifically, the imaging unit 140 is a camera or the like that can capture the capture target in the form of a moving image or a still image. More specifically, the imaging unit 140 includes a plurality of light receiving elements arranged in a two dimensional manner, and can obtain an image signal representing an image of the capture target by photoelectric conversion in the light receiving elements. The imaging unit 140 transmits the acquired image signal to the display device 30.

Note that, as in the case of the support arm device 400 illustrated in FIG. 3, the imaging unit 423 is provided at the distal end of the arm unit 420, the imaging unit 140 is actually provided at the distal end of the arm unit 120 in the robot arm device 10. FIG. 5 illustrates a state in which the imaging unit 140 is provided at a distal end of a final link via the plurality of joint units 130 and the plurality of links by schematically illustrating a link between the joint unit 130 and the imaging unit 140.

Note that, in the present embodiment, various medical instruments can be connected to the distal end of the arm unit 120 as the distal end unit. Examples of the medical instruments include various treatment instruments such as a scalpel and forceps, and various units used in treatment, such as a unit of various detection devices such as probes of an ultrasonic examination device. Furthermore, in the present embodiment, the imaging unit 140 illustrated in FIG. 5 or a unit having an imaging function such as an endoscope or a microscope may also be included in the medical instruments. Thus, the arm device 10 according to the present embodiment can be said to be a medical arm device provided with medical instruments. Similarly, the arm control system 1 according to the present embodiment can be said to be a medical arm control system. Note that the arm device 10 illustrated in FIG. 5 can also be said to be a scope holding arm device provided with a unit having an imaging function as the distal end unit. Furthermore, a stereo camera having two imaging units (camera units) may be provided at the distal end of the arm unit 120, and may capture an imaging target to be displayed as a 3D image.

The function and configuration of the arm device 10 have been described above. Next, a function and a configuration of the control device 20 will be described. Referring to FIG. 5, the control device 20 includes an input unit 210, a storage unit 220, and a control unit 230.

The control unit 230 integrally controls the control device 20 and performs various operations for controlling the driving of the arm unit 120 in the arm device 10. Specifically, to control the driving of the arm unit 120 of the arm device 10, the control unit 230 performs various operations in the whole body coordination control and the ideal joint control. Hereinafter, the function and configuration of the control unit 230 will be described in detail. The whole body coordination control and the ideal joint control have been already described in <2-2. Generalized Inverse Dynamics> and <2-3. Ideal Joint Control> above, and thus detailed description is omitted here.

The control unit 230 includes a whole body coordination control unit 240 and an ideal joint control unit 250.

The whole body coordination control unit 240 performs various operations regarding the whole body coordination control using the generalized inverse dynamics. In the present embodiment, the whole body coordination control unit 240 acquires a state (arm state) of the arm unit 120 on the basis of the state of the joint unit 130 detected by the joint state detection unit 132. Furthermore, the whole body coordination control unit 240 calculates a control value for the whole body coordination control of the arm unit 120 in an operation space, using the generalized inverse dynamics, on the basis of the arm state, and a motion purpose and a constraint condition of the arm unit 120. Note that the operation space is a space for describing the relationship between the force acting on the arm unit 120 and the acceleration generated in the arm unit 120, for example.

The whole body coordination control unit 240 includes an arm state acquisition unit 241, an arithmetic condition setting unit 242, a virtual force calculation unit 243, and a real force calculation unit 244.

The arm state acquisition unit 241 acquires the state (arm state) of the arm unit 120 on the basis of the state of the joint unit 130 detected by the joint state detection unit 132. Here, the arm state may mean the state of motion of the arm unit 120. For example, the arm state includes information such as the position, speed, acceleration, and force of the arm unit 120. As described above, the joint state detection unit 132 acquires, as the state of the joint unit 130, the information of the rotation angle, rotation angular speed, rotation angular acceleration, generated torque, and the like in each joint unit 130. Furthermore, although to be described below, the storage unit 220 stores various types of information to be processed by the control device 20. In the present embodiment, the storage unit 220 may store various types of information (arm information) regarding the arm unit 120, for example, the number of joint units 130 and links configuring the arm unit 120, connection states between the links and the joint units 130, and lengths of the links, and the like. The arm state acquisition unit 241 can acquire the arm information from the storage unit 220. Therefore, the arm state acquisition unit 241 can acquire, as the arm state, information such as the positions (coordinates) in the space of the plurality of joint units 130, the plurality of links, and the imaging unit 140 (in other words, the shape of the arm unit 120 and the position and posture of the imaging unit 140), and the forces acting on the joint units 130, the links, and the imaging unit 140, on the basis of the state and the arm information of the joint units 130. The arm state acquisition unit 241 transmits the acquired arm information to the arithmetic condition setting unit 242.

The arithmetic condition setting unit 242 sets operation conditions in an operation regarding the whole body coordination control using the generalized inverse dynamics. Here, the operation condition may be a the motion purpose and a constraint condition. The motion purpose may be various types of information regarding the motion of the arm unit 120. Specifically, the motion purpose may be target values of the position and posture (coordinates), speed, acceleration, force, and the like of the imaging unit 140, or target values of the positions (coordinates), speeds, accelerations, forces, and the like of the plurality of joint units 130 and the plurality of links of the arm unit 120. Furthermore, the constraint condition may be various types of information that restricts (restrains) the motion of the arm unit 120. Specifically, the constraint condition may be coordinates of a region where each configuration component of the arm unit cannot move, an unmovable speed, a value of acceleration, a value of an ungenerable force, and the like. Furthermore, restriction ranges of various physical quantities under the constraint condition may be set according to inability to structurally realizing the arm unit 120 or may be appropriately set by the user. Furthermore, the arithmetic condition setting unit 242 includes a physical model for the structure of the arm unit 120 (in which, for example, the number and lengths of the links configuring the arm unit 120, the connection states of the links via the joint units 130, the movable ranges of the joint units 130, and the like are modeled), and may set a motion condition and the constraint condition by generating a control model in which the desired motion condition and constraint condition are reflected in the physical model.

In the present embodiment, appropriate setting of the motion purpose and the constraint condition enables the arm unit 120 to perform a desired operation. For example, not only can the imaging unit 140 be moved to a target position by setting a target value of the position of the imaging unit 140 as the motion purpose but also the arm unit 120 can be driven by providing a constraint of movement by the constraint condition to prevent the arm unit 120 from intruding into a predetermined region in the space.

A specific example of the motion purpose includes, for example, a pivot operation, which is a turning operation with an axis of a cone serving as a pivot axis, in which the imaging unit 140 moves in a conical surface setting an operation site as a top in a state where the capture direction of the imaging unit 140 is fixed to the operation site. Furthermore, in the pivot operation, the turning operation may be performed in a state where the distance between the imaging unit 140 and a point corresponding to the top of the cone is kept constant. By performing such a pivot operation, an observation site can be observed from an equal distance and at different angles, whereby the convenience of the user who performs surgery can be improved.

Furthermore, as another specific example, the motion purpose may be content to control the generated torque in each joint unit 130. Specifically, the motion purpose may be a power assist operation to control the state of the joint unit 130 to cancel the gravity acting on the arm unit 120, and further control the state of the joint unit 130 to support the movement of the arm unit 120 in a direction of a force provided from the outside. More specifically, in the power assist operation, the driving of each joint unit 130 is controlled to cause each joint unit 130 to generate a generated torque that cancels the external torque due to the gravity in each joint unit 130 of the arm unit 120, whereby the position and posture of the arm unit 120 are held in a predetermined state. In a case where an external torque is further added from the outside (for example, from the user) in the aforementioned state, the driving of each joint unit 130 is controlled to cause each joint unit 130 to generate a generated torque in the same direction as the added external torque. By performing such a power assist operation, the user can move the arm unit 120 with a smaller force in a case where the user manually moves the arm unit 120. Therefore, a feeling as if the user moved the arm unit 120 under weightlessness can be provided to the user. Furthermore, the above-described pivot operation and the power assist operation can be combined.

Here, in the present embodiment, the motion purpose may mean an operation (motion) of the arm unit 120 realized by the whole body coordination control or may mean an instantaneous motion purpose in the operation (in other words, a target value in the motion purpose). For example, in the above-described pivot operation, the imaging unit 140 performing the pivot operation itself is the motion purpose. In the act of performing the pivot operation, values of the position, speed, and the like of the imaging unit 140 in a conical surface in the pivot operation are set as the instantaneous motion purpose (the target values in the motion purpose). Furthermore, in the above-described power assist operation, for example, performing the power assist operation to support the movement of the arm unit 120 in the direction of the force applied from the outside itself is the motion purpose. In the act of performing the power assist operation, the value of the generated torque in the same direction as the external torque applied to each joint unit 130 is set as the instantaneous motion purpose (the target value in the motion purpose). The motion purpose in the present embodiment is a concept including both the instantaneous motion purpose (for example, the target values of the positions, speeds, forces, and the like of the configuration members of the arm unit 120 at a certain time) and the operations of the configuration members of the arm unit 120 realized over time as a result of the instantaneous motion purpose having been continuously achieved. The instantaneous motion purpose is set each time in each step in an operation for the whole body coordination control in the whole body coordination control unit 240, and the operation is repeatedly performed, so that the desired motion purpose is finally achieved.

Note that, in the present embodiment, the viscous drag coefficient in a rotation motion of each joint unit 130 may be appropriately set when the motion purpose is set. As described above, the joint unit 130 according to the present embodiment is configured to be able to appropriately adjust the viscous drag coefficient in the rotation motion of the actuator. Therefore, by setting the viscous drag coefficient in the rotation motion of each joint unit 130 when setting the motion purpose, an easily rotatable state or a less easily rotatable state can be realized for the force applied from the outside, for example. For example, in the above-descried power assist operation, when the viscous drag coefficient in the joint unit 130 is set to be small, a force required by the user to move the arm unit 120 can be made small, and a weightless feeling provided to the user can be promoted. As described above, the viscous drag coefficient in the rotation motion of each joint unit 130 may be appropriately set according to the content of the motion purpose.

Here, in the present embodiment, as will be described below, the storage unit 220 may store parameters regarding the operation conditions such as the motion purpose and the constraint condition used in the operation regarding the whole body coordination control. The arithmetic condition setting unit 242 can set the constraint condition stored in the storage unit 220 as the constraint condition used for the operation of the whole body coordination control.

Furthermore, in the present embodiment, the arithmetic condition setting unit 242 can set the motion purpose by a plurality of methods. For example, the arithmetic condition setting unit 242 may set the motion purpose on the basis of the arm state transmitted from the arm state acquisition unit 241. As described above, the arm state includes information of the position of the arm unit 120 and information of the force acting on the arm unit 120. Therefore, for example, in a case where the user is trying to manually move the arm unit 120, information regarding how the user is moving the arm unit 120 is also acquired by the arm state acquisition unit 241 as the arm state. Therefore, the arithmetic condition setting unit 242 can set the position, speed, force, and the like to/at/with which the user has moved the arm unit 120, as the instantaneous motion purpose, on the basis of the acquired arm state. By thus setting the motion purpose, the driving of the arm unit 120 is controlled to follow and support the movement of the arm unit 120 by the user.

Furthermore, for example, the arithmetic condition setting unit 242 may set the motion purpose on the basis of an instruction input from the input unit 210 by the user. Although to be described below, the input unit 210 is an input interface for the user to input information, commands, and the like regarding the drive control of the arm device 10, to the control device 20. In the present embodiment, the motion purpose may be set on the basis of an operation input from the input unit 210 by the user. Specifically, the input unit 210 has, for example, operation means operated by the user, such as a lever and a pedal. The positions, speeds, and the like of the configuration members of the arm unit 120 may be set as the instantaneous motion purpose by the arithmetic condition setting unit 242 in response to an operation of the lever, pedal, or the like.

Moreover, for example, the arithmetic condition setting unit 242 may set the motion purpose stored in the storage unit 220 as the motion purpose used for the operation of the whole body coordination control. For example, in the case of the motion purpose that the imaging unit 140 stands still at a predetermined point in the space, coordinates of the predetermined point can be set in advance as the motion purpose. Furthermore, for example, in the case of the motion purpose that the imaging unit 140 moves on a predetermined trajectory in the space, coordinates of each point representing the predetermined trajectory can be set in advance as the motion purpose. As described above, in a case where the motion purpose can be set in advance, the motion purpose may be stored in the storage unit 220 in advance. Furthermore, in the case of the above-described pivot operation, for example, the motion purpose is limited to a motion purpose setting the position, speed, and the like in the conical surface as the target values. In the case of the power assist operation, the motion purpose is limited to a motion purpose setting the force as the target value. In the case where the motion purpose such as the pivot operation or the power assist operation is set in advance in this way, information regarding ranges, types and the like of the target values settable as the instantaneous motion purpose in such a motion purpose may be stored in the storage unit 220. The arithmetic condition setting unit 242 can also set the various types of information regarding such a motion purpose as the motion purpose.

Note that by which method the arithmetic condition setting unit 242 sets the motion purpose may be able to be appropriately set by the user according to the application of the arm device 10 or the like. Furthermore, the arithmetic condition setting unit 242 may set the motion purpose and the constraint condition by appropriately combining the above-described methods. Note that a priority of the motion purpose may be set in the constraint condition stored in the storage unit 220, or in a case where is a plurality of motion purposes different from one another, the arithmetic condition setting unit 242 may set the motion purpose according to the priority of the constraint condition. The arithmetic condition setting unit 242 transmits the arm state and the set motion purpose and constraint condition to the virtual force calculation unit 243.

The virtual force calculation unit 243 calculates a virtual force in the operation regarding the whole body coordination control using the generalized inverse dynamics. The processing of calculating the virtual force performed by the virtual force calculation unit 243 may be the series of processing described in, for example, <2-2-1. Virtual Force Calculation Processing> above. The virtual force calculation unit 243 transmits the calculated virtual force fᵥ to the real force calculation unit 244.

The real force calculation unit 244 calculates a real force in the operation regarding the whole body coordination control using the generalized inverse dynamics. The processing of calculating the real force performed by the real force calculation unit 244 may be the series of processing described in, for example, <2-2-2. Real Force Calculation Processing> above. The real force calculation unit 244 transmits the calculated real force (generated torque) τₐ to the ideal joint control unit 250. Note that, in the present embodiment, the generated torque τₐ calculated by the real force calculation unit 244 is also referred to as a control value or a control torque value in the sense of a control value of the joint unit 130 in the whole body coordination control.

The ideal joint control unit 250 performs various operations regarding the ideal joint control using the generalized inverse dynamics. In the present embodiment, the ideal joint control unit 250 correct the influence of disturbance for the generated torque τₐ calculated by the real force calculation unit 244 to calculate a torque command value τ realizing an ideal response of the arm unit 120. Note that the arithmetic processing performed by the ideal joint control unit 250 corresponds to the series of processing described in <2-3. Ideal Joint Control> above.

The ideal joint control unit 250 includes a disturbance estimation unit 251 and a command value calculation unit 252.

The disturbance estimation unit 251 calculates a disturbance estimation value τ_{d} on the basis of the torque command value τ and the rotation angular speed calculated from the rotation angle q detected by the rotation angle detection unit 133. Note that the torque command value τ mentioned here is a command value that represents the generated torque in the arm unit 120 to be finally transmitted to the arm device 10. Thus, the disturbance estimation unit 251 has a function corresponding to the disturbance observer 620 illustrated in FIG. 4.

The command value calculation unit 252 calculates the torque command value τ that is a command value representing the torque to be generated in the arm unit 120 and finally transmitted to the arm device 10, using the disturbance estimation value τ_{d} calculated by the disturbance estimation unit 251. Specifically, the command value calculation unit 252 adds the disturbance estimation value I_{d} calculated by the disturbance estimation unit 251 to τ^{ref} calculated from the ideal model of the joint unit 130 described in the above expression (12) to calculate the torque command value τ. For example, win a case where the disturbance estimation value τ_{d} is not calculated, the torque command value τ becomes the torque target value τ^{ref}. Thus, the function of the command value calculation unit 252 corresponds to the function other than the disturbance observer 620 illustrated in FIG. 4.

As described above, in the ideal joint control unit 250, the information is repeatedly exchanged between the disturbance estimation unit 251 and the command value calculation unit 252, so that the series of processing described with reference to FIG. 4 is performed. The ideal joint control unit 250 transmits the calculated torque command value τ to the drive control unit 111 of the arm device 10. The drive control unit 111 performs control to supply the current amount corresponding to the transmitted torque command value τ to the motor in the actuator of the joint unit 130, thereby controlling the number of rotations of the motor and controlling the rotation angle and the generated torque in the joint unit 130.

In the arm control system 1 according to the present embodiment, the drive control of the arm unit 120 in the arm device 10 is continuously performed during work using the arm unit 120, so the above-described processing in the arm device 10 and the control device 20 is repeatedly performed. In other words, the state of the joint unit 130 is detected by the joint state detection unit 132 of the arm device 10 and transmitted to the control device 20. The control device 20 performs various operations regarding the whole body coordination control and the ideal joint control for controlling the driving of the arm unit 120 on the basis of the state of the joint unit 130, and the motion purpose and the constraint condition, and transmits the torque command value τ as the operation result to the arm device 10. The arm device 10 controls the driving of the arm unit 120 on the basis of the torque command value τ, and the state of the joint unit 130 during or after the driving is detected by the joint state detection unit 132 again.

Description about other configurations included in the control device 20 will be continued.

The input unit 210 is an input interface for the user to input information, commands, and the like regarding the drive control of the arm device 10 to the control device 20. In the present embodiment, the driving of the arm unit 120 of the arm device 10 may be controlled on the basis of the operation input from the input unit 210 by the user, and the position and posture of the imaging unit 140 may be controlled. Specifically, as described above, instruction information regarding the instruction of the driving of the arm input from the input unit 210 by the user is input to the arithmetic condition setting unit 242, so that the arithmetic condition setting unit 242 may set the motion purpose in the whole body coordination control on the basis of the instruction information. The whole body coordination control is performed using the motion purpose based on the instruction information input by the user as described above, so that the driving of the arm unit 120 according to the operation input of the user is realized.

Specifically, the input unit 210 includes operation means operated by the user, such as a mouse, a keyboard, a touch panel, a button, a switch, a lever, and a pedal, for example. For example, in a case where the input unit 210 has a pedal, the user can control the driving of the arm unit 120 by operating the pedal with the foot. Therefore, even in a case where the user is performing treatment using both hands on the operation site of the patient, the user can adjust the position and posture of the imaging unit 140, in other words, the user can adjust a capture position and a capture angle of the operation site, by the operation of the pedal with the foot.

The storage unit 220 stores various types of information processed by the control device 20. In the present embodiment, the storage unit 220 can store various parameters used in the operation regarding the whole body coordination control and the ideal joint control performed by the control unit 230. For example, the storage unit 220 may store the motion purpose and the constraint condition used in the operation regarding the whole body coordination control by the whole body coordination control unit 240. The motion purpose stored in the storage unit 220 may be, as described above, a motion purpose that can be set in advance, such as, for example, the imaging unit 140 standing still at a predetermined point in the space. Furthermore, the constraint conditions may be set in advance by the user and stored in the storage unit 220 according to a geometric configuration of the arm unit 120, the application of the robot arm device 10, and the like. Furthermore, the storage unit 220 may also store various types of information regarding the arm unit 120 used when the arm state acquisition unit 241 acquires the arm state. Moreover, the storage unit 220 may store the operation result, various numerical values, and the like calculated in the operation process in the operation regarding the whole body coordination control and the ideal joint control by the control unit 230. As described above, the storage unit 220 may store any parameters regarding the various types of processing performed by the control unit 230, and the control unit 230 can performs various types of processing while mutually exchanging information with the storage unit 220.

The function and configuration of the control device 20 have been described above. Note that the control device 20 according to the present embodiment can be configured by, for example, various information processing devices (arithmetic processing devices) such as a personal computer (PC) and a server. Next, a function and a configuration of the display device 30 will be described.

The display device 30 displays the information on the display screen in various formats such as texts and images to visually notify the user of various types of information. In the present embodiment, the display device 30 displays the image captured by the imaging unit 140 of the arm device 10 on the display screen. Specifically, the display device 30 has functions and configurations of an image signal processing unit (not illustrated) that applies various types of image processing to an image signal acquired by the imaging unit 140, a display control unit (not illustrated) that performs control to display an image based on the processed image signal on the display screen, and the like. Note that the display device 30 may have various functions and configurations that a display device generally has, in addition to the above-described functions and configurations. The display device 30 corresponds to the display device 5041 illustrated in FIG. 1.

The functions and configurations of the arm device 10, the control device 20, and the display device 30 according to the present embodiment have been described above with reference to FIG. 5. Each of the above-described constituent elements may be configured using general-purpose members or circuit, or may be configured by hardware specialized for the function of each constituent element. Furthermore, all the functions of the configuration elements may be performed by a CPU or the like. Therefore, the configuration to be used can be changed as appropriate according to the technical level of the time of carrying out the present embodiment.

As described above, according to the present embodiment, the arm unit 120 that is the multilink structure in the arm device 10 has at least six degrees or more of freedom, and the driving of each of the plurality of joint units 130 configuring the arm unit 120 is controlled by the drive control unit 111. Then, a medical instrument is provided at the distal end of the arm unit 120. The driving of each of the joint units 130 is controlled as described above, so that the drive control of the arm unit 120 with a higher degree of freedom is realized, and the medical arm device 10 with higher operability for the user is realized.

More specifically, according to the present embodiment, the joint state detection unit 132 detects the state of the joint unit 130 in the arm device 10. Then, the control device 20 performs various operations regarding the whole body coordination control using the generalized inverse dynamics for controlling the driving of the arm unit 120 on the basis of the state of the joint unit 130, and the motion purpose and the constraint condition, and calculates the torque command value τ as the operation result. Moreover, the arm device 10 controls the driving of the arm unit 120 on the basis of the torque command value τ. As described above, in the present embodiment, the driving of the arm unit 120 is controlled by the whole body coordination control using the generalized inverse dynamics. Therefore, the drive control of the arm unit 120 by force control is realized, and an arm device with higher operability for the user is realized. Furthermore, in the present embodiment, control to realize various motion purposes for further improving the convenience of the user, such as the pivot operation and the power assist operation, is possible in the whole body coordination control. Moreover, in the present embodiment, various driving means are realized, such as manually moving the arm unit 120, and moving the arm unit 120 by the operation input from a pedal. Therefore, further improvement of the convenience for the user is realized.

Furthermore, in the present embodiment, the ideal joint control is applied together with the whole body coordination control to the drive control of the arm unit 120. In the ideal joint control, the disturbance components such as friction and inertia inside the joint unit 130 are estimated, and the feedforward control using the estimated disturbance components is performed. Therefore, even in a case where there is a disturbance component such as friction, an ideal response can be realized for the driving of the joint unit 130. Therefore, in the drive control of the arm unit 120, highly accurate response and high positioning accuracy and stability with less influence of vibration and the like are realized.

Moreover, in the present embodiment, each of the plurality of joint units 130 configuring the arm unit 120 has a configuration adapted to the ideal joint control, and the rotation angle, generated torque and viscous drag coefficient in each joint unit 130 can be controlled with the current value. As described above, the driving of each joint unit 130 is controlled with the current value, and the driving of each joint unit 130 is controlled while grasping the state of the entire arm unit 120 by the whole body coordination control. Therefore, counterbalance is unnecessary and downsizing of the arm device 10 is realized.

### 3. Configuration Example of Microsurgical System

The technology according to the present disclosure can be applied to various products. For example, the technology according to the present disclosure may be applied to a microsurgical system used for so-called microsurgery, which is performed while performing close-up observation of a patient's minute site.

FIG. 6 is a view illustrating an example of a schematic configuration of a microsurgical system 5300 to which the technology according to the present disclosure is applicable. Referring to FIG. 6, the microsurgical system 5300 includes a microscope device 5301, a control device 5317, and a display device 5319. Note that, in the following description of the microsurgical system 5300, “user” means any medical staff who uses the microsurgical system 5300, such as an operator or an assistant.

The microscope device 5301 includes a microscope unit 5303 for magnifying and observing an observation target (an operation site of a patient), an arm unit 5309 supporting the microscope unit 5303 at a distal end, and a base unit 5315 supporting a proximal end of the arm unit 5309.

The microscope unit 5303 includes a substantially cylindrical tubular portion 5305, an imaging unit (not illustrated) provided inside the tubular portion 5305, and an operation portion 5307 provided in a partial region of an outer periphery of the tubular portion 5305. The microscope unit 5303 is an electronic imaging microscope unit (so-called video microscope unit) that electronically captures a captured image by the imaging unit.

A cover glass for protecting the imaging unit inside is provided on an opening surface in a lower end of the tubular portion 5305. Light from the observation target (hereinafter, also referred to as observation light) passes through the cover glass and enters the imaging unit inside the tubular portion 5305. Note that a light source including, for example, a light emitting diode (LED) and the like may be provided inside the tubular portion 5305, and the observation target may be irradiated with light from the light source via the cover glass at the time of imaging.

The imaging unit includes an optical system that condenses the observation light and an imaging element that receives the observation light condensed by the optical system. The optical system is configured by a combination of a plurality of lenses including a zoom lens and a focus lens, and optical characteristics of the optical system are adjusted so as to focus the observation light on a light receiving surface of the imaging element. The imaging element receives and photoelectrically converts the observation light to generate a signal corresponding to the observation light, in other words, an image signal corresponding to an observed image. As the imaging element, for example, an imaging element capable of capturing a color image including a Bayer array is used. The imaging element may be various known imaging elements such as a complementary metal oxide semiconductor (CMOS) image sensor or a charge coupled device (CCD) image sensor. The image signal generated by the imaging element is transmitted to the control device 5317 as raw data. Here, the transmission of the image signal may be suitably performed by optical communication. This is because, in a surgical site, the operator performs surgery while observing a state of an affected part with the captured image, and thus display of a moving image of an operation site in as real time as possible is demanded for more safe and reliable surgery. When the image signal is transmitted by optical communication, the captured image becomes able to be displayed with low latency.

Note that the imaging unit may have a drive mechanism that moves the zoom lens and the focus lens of the optical system along an optical axis. By appropriately moving the zoom lens and the focus lens by the drive mechanism, magnification and a focal length at the time of imaging of the captured image can be adjusted. Furthermore, the imaging unit may be equipped with various functions that can be generally provided in an electronic imaging microscope unit, such as an auto exposure (AE) function and an auto focus (AF) function.

Furthermore, the imaging unit may be configured as a so-called single-plate imaging unit including one imaging device, or may be configured as a so-called multi-plate imaging unit including a plurality of imaging elements. In a case where the imaging unit is configured as a multi-plate imaging unit, for example, a color image may be obtained by generating image signals respectively corresponding to RGB by the imaging elements and synthesizing the image signals. Alternatively, the imaging unit may include a pair of imaging elements for respectively obtaining image signals for right eye and for left eye corresponding to three-dimensional (3D) display. With the 3D display, the operator can more accurately grasp the depth of biological tissue in the operation site. Note that, in a case where the imaging unit is configured as a multi-plate imaging unit, a plurality of systems of the optical system can be provided corresponding to the imaging elements.

The operation portion 5307 includes, for example, a cross lever, a switch, or the like, and is input means that receives a user's operation input. For example, the user can input an instruction to change the magnification of the observed image and the focal length to the observation target via the operation portion 5307. By appropriately moving the zoom lens and the focus lens by the drive mechanism of the imaging unit according to the instruction, the magnification and the focal length can be adjusted. Furthermore, for example, the user can input an instruction to switch an operation mode (all free mode and fixed mode described below) of the arm unit 5309 via the operation portion 5307. Note that, in a case where the user tries to move the microscope unit 5303, a mode in which the user moves the microscope unit 5303 in a state of holding the tubular portion 5305 is assumed. Therefore, it is favorable to provide the operation portion 5307 at a position where the user can easily operate the operation portion 5307 with a finger in a state of holding the tubular portion 5305 so that the user can operate the operation portion 5307 while moving the tubular portion 5305.

The arm unit 5309 is configured such that a plurality of links (first link 5313a to sixth link 5313f) is rotatably connected with one another by a plurality of joint units (first joint unit 5311a to sixth joint unit 5311f).

The first joint unit 5311a has a substantially cylindrical shape, and a distal end (lower end) of the first joint unit 5311a rotatably supports an upper end of the tubular portion 5305 of the microscope unit 5303 around a rotation axis (first axis O₁) parallel to a central axis of the tubular portion 5305. Here, the first joint unit 5311a can be configured such that the first axis O₁ coincides with an optical axis of the imaging unit of the microscope unit 5303. With the configuration, a field of view can be changed to rotate a captured image by rotating the microscope unit 5303 around the first axis O₁.

The first link 5313a fixedly supports the first joint unit 5311a at the distal end. Specifically, the first link 5313a is a rod-like member having a substantially L-shape, and is connected to the first joint unit 5311a such that an end portion of one side on a distal end side comes in contact with an upper end portion of an outer periphery of the first joint unit 5311a while the one side on the distal end side extends in a direction orthogonal to the first axis O₁. The second joint unit 5311b is connected to an end portion of the other side on a proximal end side of the substantially L-shape of the first link 5313a.

The second joint unit 5311b has a substantially cylindrical shape, and a distal end of the second joint unit 5311b rotatably supports the proximal end of the first link 5313a around a rotation axis (second axis O₂) orthogonal to the first axis O₁. A distal end of the second link 5313b is fixedly connected to a proximal end of the second joint unit 5311b.

The second link 5313b is a rod-like member having a substantially L-shape, and an end portion of one side on a distal end side is fixedly connected to the proximal end of the second joint unit 5311b while the one side extends in a direction orthogonal to the second axis O₂. The third joint unit 5311c is connected to the other side on a proximal end side of the substantially L-shape of the second link 5313b.

The third joint unit 5311c has a substantially cylindrical shape, and a distal end of the third joint unit 5311c rotatably supports the proximal end of the second link 5313b around a rotation axis (third axis O₃) orthogonal to the first axis O₁ and the second axis O₂. A distal end of the third link 5313c is fixedly connected to a proximal end of the third joint unit 5311c. The microscope unit 5303 can be moved to change the position of the microscope unit 5303 in a horizontal plane by rotating the configuration on the distal end side including the microscope unit 5303 around the second axis O₂ and the third axis O₃. That is, by controlling the rotation around the second axis O₂ and the third axis O₃, the field of view of the captured image can be moved in the plane.

The third link 5313c is configured such that its distal end side has a substantially cylindrical shape, and the proximal end of the third joint unit 5311c is fixedly connected to the distal end of the cylindrical shape such that both the third joint unit 5311c and the cylindrical shape have substantially the same central axis. A proximal end side of the third link 5313c has a prismatic shape, and the fourth joint unit 5311d is connected to an end portion on the proximal end side.

The fourth joint unit 5311d has a substantially cylindrical shape, and a distal end of the fourth joint unit 5311d rotatably supports the proximal end of the third link 5313c around a rotation axis (fourth axis O₄) orthogonal to the third axis O₃ A distal end of the fourth link 5313d is fixedly connected to a proximal end of the fourth joint unit 5311d.

The fourth link 5313d is a substantially linearly extending rod-like member, and is fixedly connected to the fourth joint unit 5311d while extending to be orthogonal to the fourth axis O₄ such that an end portion on a distal end of the fourth link 5313d comes in contact with a side surface of the substantially cylindrical shape of the fourth joint unit 5311d. The fifth joint unit 5311e is connected to a proximal end of the fourth link 5313d.

The fifth joint unit 5311e has a substantially cylindrical shape, and a distal end of the fifth joint unit 5311e rotatably supports the proximal end of the fourth link 5313d around a rotation axis (fifth axis O₅) parallel to the fourth axis O₄. A distal end of the fifth link 5313e is fixedly connected to a proximal end of the fifth joint unit 5311e. The fourth axis O₄ and the fifth axis O₅ are rotation axes enabling the microscope unit 5303 to move in the up-down direction. The height of the microscope unit 5303, in other words, the distance between the microscope unit 5303 and the observation target can be adjusted by rotating the configuration on the distal end side including the microscope unit 5303 around the fourth axis O₄ and the fifth axis O₅.

The fifth link 5313e is configured by a combination of a first member having a substantially L-shape and having one side extending in a vertical direction and the other side extending in a horizontal direction, and a rod-like second member extending vertically downward from a portion of the first member extending in the horizontal direction. The proximal end of the fifth joint unit 5311e is fixedly connected to a vicinity of an upper end of the portion of the first member of the fifth link 5313e, the portion extending in the vertical direction. The sixth joint unit 5311f is connected to a proximal end (lower end) of the second member of the fifth link 5313e.

The sixth joint unit 5311f has a substantially cylindrical shape, and a distal end of the sixth joint unit 5311f rotatably supports the proximal end of the fifth link 5313e around a rotation axis (sixth axis O₆) parallel to the vertical direction. A distal end of the sixth link 5313f is fixedly connected to a proximal end of the sixth joint unit 5311f.

The sixth link 5313f is a rod-like member extending in the vertical direction, and the proximal end of the sixth link 5313f is fixedly connected to an upper surface of the base unit 5315.

Rotatable ranges of the first joint unit 5311a to sixth joint unit 5311f are appropriately set such that the microscope unit 5303 can perform desired movement. Thereby, in the arm unit 5309 having the above-described configuration, the movement of a total of six degrees of freedom, i.e., three translational degrees of freedom and three rotational degrees of freedom, can be realized with respect to the movement of the microscope unit 5303. By configuring the arm unit 5309 to realize six degrees of freedom with respect to the movement of the microscope unit 5303 in this way, the position and posture of the microscope unit 5303 can be freely controlled within the movable range of the arm unit 5309. Therefore, the operation site can be observed from any angle, and the surgery can be more smoothly executed.

Note that the configuration of the illustrated arm unit 5309 is merely an example, and the number and shapes (lengths) of the links configuring the arm unit 5309, and the number, arrangement positions, directions of the rotation axes, and the like of the joint units may be appropriately designed to realize desired degrees of freedom. For example, as described above, to freely move the microscope unit 5303, the arm unit 5309 is favorably configured to have six degrees of freedom. However, the arm unit 5309 may be configured to have larger degrees of freedom (in other words, redundant degrees of freedom). In a case where the redundant degrees of freedom exist, in the arm unit 5309, the posture of the arm unit 5309 can be changed in a case where the position and posture of the microscope unit 5303 are fixed. Therefore, more convenient control for the operator can be realized, such as controlling the posture of the arm unit 5309 such that the arm unit 5309 does not interfere with the field of vision of the operator looking at the display device 5319, for example.

Here, each of the first joint unit 5311a to sixth joint unit 5311f can be provided with an actuator mounted with a drive mechanism such as a motor, an encoder for detecting the rotation angle in each joint unit, and the like. Then, the posture of the arm unit 5309, in other words, the position and posture of the microscope unit 5303 can be controlled as driving of the actuators provided in the first joint unit 5311a to sixth joint unit 5311f are appropriately controlled by the control device 5317. Specifically, the control device 5317 can grasp the current posture of the arm unit 5309, and the current position and posture of the microscope unit 5303, on the basis of information of the rotation angles of the joint units detected by the encoders. The control device 5317 calculates control values (for example, rotation angles or generated torques, and the like) for the joint units that realize the movement of the microscope unit 5303 according to the operation input from the user, using the grasped information grasped, and drives the drive mechanisms of the joint units according to the control values. Note that, at this time, the method of controlling the arm unit 5309 by the control device 5317 is not limited, and various known control methods such as force control or position control may be applied.

For example, the driving of the arm unit 5309 may be appropriately controlled by the control device 5317 according to the operation input, and the position and posture of the microscope unit 5303 may be controlled by the operator appropriately performing an operation input via an input device (not illustrated). With the control, the microscope unit 5303 can be moved from an arbitrary position to an arbitrary position, and then can be fixedly supported at the position after the movement. Note that, as the input device, an input device operable by the operator, such as a foot switch, for example, even if the operator holds a treatment tool, is favorably applied. Furthermore, the operation input may be performed in a non-contact manner on the basis of gesture detection or gaze detection using a camera provided on a wearable device or in the operating room. As a result, even a user belonging to a clean area can operate devices belonging to a filthy area with a higher degree of freedom. Alternatively, the arm unit 5309 may be operated by a so-called master-slave system. In this case, the arm unit 5309 can be remotely operated by the user via the input device installed at a place distant from an operating room.

Furthermore, in a case where the force control is applied, so-called power assist control in which an external force is received from the user, and the actuators of the first joint unit 5311a to sixth joint unit 5311f are driven such that the arm unit 5309 smoothly moves along the external force may be performed. With the control, the user can move the microscope unit 5303 with a relatively light force when directly moving while holding the microscope unit 5303 to another position. Accordingly, the user can more intuitively move the microscope unit 5303 with a simpler operation, and the user's convenience can be improved.

Furthermore, the driving of the arm unit 5309 may be controlled to perform a pivot operation. Here, the pivot operation is an operation to move the microscope unit 5303 such that the optical axis of the microscope unit 5303 always faces a predetermined point in the space (hereinafter, referred to as a pivot point). According to the pivot operation, the same observation position can be observed from various directions, and more detailed observation of the affected part becomes possible. Note that, in a case where the microscope unit 5303 is configured to be unable to adjust its focal length, it is favorably to perform the pivot operation in a case where the distance between the microscope unit 5303 and the pivot point is fixed. In this case, it is sufficient that the distance between the microscope unit 5303 and the pivot point is adjusted to a fixed focal length of the microscope unit 5303. With the adjustment, the microscope unit 5303 moves on a hemispherical surface (schematically illustrated in FIG. 6) having a radius corresponding to the focal length centered on the pivot point, and a clear captured image can be obtained even if the observation direction is changed. Meanwhile, in a case where the microscope unit 5303 is configured to be able to adjust its focal length, the pivot operation may be performed in a case where the distance between the microscope unit 5303 and the pivot point is variable. In this case, for example, the control device 5317 calculates the distance between the microscope unit 5303 and the pivot point on the basis of the information of the rotation angles of the joint units detected by the encoders, and may automatically adjusted the focal length of the microscope unit 5303 on the basis of the calculation result. Alternatively, in a case where the microscope unit 5303 is provided with an AF function, the focal length may be automatically adjusted by the AF function each time the distance between the microscope unit 5303 and the pivot point changes due to the pivot operation.

Furthermore, the first joint unit 5311a to sixth joint unit 5311f may be provided with brakes that restrict the rotation thereof. Operations of the brakes may be controlled by the control device 5317. For example, in a case where it is desired to fix the position and posture of the microscope unit 5303, the control device 5317 actuates the brakes of the respective joint units. Accordingly, the posture of the arm unit 5309, in other words, the position and posture of the microscope unit 5303 can be fixed even if the actuators are not driven. Therefore, the power consumption can be decreased. In a case where it is desired to move the position and posture of the microscope unit 5303, it is sufficient that the control device 5317 releases the brakes of the joint units and drives the actuators according to a predetermined control method.

Such an operation of the brakes can be performed in response to an operation input by the user via the above-described operation portion 5307. In a case where the user wants to move the position and posture of the microscope unit 5303, the user operates the operation portion 5307 to release the brakes of the respective joint units. As a result, the operation mode of the arm unit 5309 shifts to a mode (all free mode) in which rotation in the joint units can be freely performed. Furthermore, in a case where the user wants to fix the position and posture of the microscope unit 5303, the user operates the operation portion 5307 to actuate the brakes of the respective joint units. As a result, the operation mode of the arm unit 5309 shifts to a mode (fixed mode) in which the rotation in the joint units is restricted.

The control device 5317 controls operations of the microscope device 5301 and the display device 5319 to centrally control the operation of the microsurgical system 5300. For example, the control device 5317 operates the actuators of the first joint unit 5311a to sixth joint unit 5311f according to a predetermined control method to control the driving of the arm unit 5309. Furthermore, for example, the control device 5317 controls the operations of the brakes of the first joint unit 5311a to sixth joint unit 5311f to change the operation mode of the arm unit 5309. Furthermore, for example, the control device 5317 applies various types of signal processing to the image signal acquired by the imaging unit of the microscope unit 5303 of the microscope device 5301 to generate image data for display and to cause the display device 5319 to display the image data. In the image processing, various types of known signal processing may be performed, such as development processing (demosaicing processing), high image quality processing (band enhancement processing, super resolution processing, noise reduction (NR) processing, and/or camera shake correction processing, for example), and/or enlargement processing (in other words, electronic zoom processing), for example.

Note that the communication between the control device 5317 and the microscope unit 5303, and the communication between the control device 5317 and the first joint unit 5311a to sixth joint unit 5311f may be wired communication or wireless communication. In the case of wired communication, communication by electrical signals may be performed or optical communication may be performed. In this case, a transmission cable used for the wired communication can be configured as an electrical signal cable, an optical fiber, or a composite cable of the aforementioned cables according to the communication system. Meanwhile, in the case of wireless communication, since it is not necessary to lay the transmission cable in the operating room, the situation in which movement of the medical staff in the operating room is hindered by the transmission cable can be resolved.

The control device 5317 can be a processor such as a central processing unit (CPU) or a graphics processing unit (GPU), a microcomputer or a control board in which a processor and a memory element such as a memory are mixed, or the like.

The processor of the control device 5317 is operated according to a predetermined program, whereby the above-described various functions can be realized. Note that, in the illustrated example, the control device 5317 is provided as a separate device from the microscope device 5301. However, the control device 5317 may be installed inside the base unit 5315 of the microscope device 5301 and integrally configured with the microscope device 5301. Alternatively, the control device 5317 may be configured by a plurality of devices. For example, microcomputers, control boards, or the like are respectively disposed in the microscope unit 5303 and the first joint unit 5311a to sixth joint unit 5311f of the arm unit 5309, and are communicatively connected to one another, whereby similar functions to the control device 5317 may be realized.

The display device 5319 is provided in the operating room, and displays an image corresponding to the image data generated by the control device 5317 under the control of the control device 5317. That is, an image of the operation site captured by the microscope unit 5303 is displayed on the display device 5319. Note that the display device 5319 may display various types of information regarding surgery, such as physical information of the patient and information regarding an operation technique of the surgery, for example, in place of or in addition to the image of the operation site. In this case, the display of the display device 5319 may be appropriately switched by the operation by the user. Alternatively, a plurality of the display devices 5319 may be provided, and the image of the operation site and the various types of information regarding surgery may be displayed on each of the plurality of display devices 5319.

Note that, as the display device 5319, various known display devices such as a liquid crystal display device or an electro luminescence (EL) display device may be applied.

### 4. Estimation of Force Acting from Disturbance According to Present Embodiment

In the present embodiment, formulation of the estimation of the force acting from the disturbance is performed by considering an actual surgical scene (including the relationship with surgical tools other than the arm or the like) and environmental conditions. Thereby, estimation of forces of various types of disturbance acting on the arm in the surgical environment can be realized. Thereby, application of safety stop by contact detection or switching of the control state of the arm by the operation force detection or the like to the user interface, and implementation of an application such as presentation of force sense to the outside become possible.

Such technology is different from the conventional technology by “(1) physical and structural characteristics of the robot”. Furthermore, such technology is different from the conventional technology by “(2) the characteristics that the external force due to main disturbance (tension due to the light source and the camera cable) is limited, and points where other types of disturbance act can be assumed”.

First, the above-described “(1) physical and structural characteristics of the robot” will be described. First, the technology according to the present embodiment is different from the conventional technology using a medical arm in that installation of a force sensor is not necessary. Furthermore, second, the technology according to the present embodiment is different from the conventional technology using a general-purpose arm in that a decrease in force estimation accuracy due to small manipulability can be avoided in a body structure assuming an operation site and an operation method.

Subsequently, “(2) the characteristics that the external force due to main disturbance (tension due to the light source and the camera cable) is limited, and points where other types of disturbance act can be assumed” will be described. According to the technology of the present embodiment, the disturbance due to the cable can be estimated, and the control and force assist for compensating the disturbance can be performed. In addition, the disturbance other than due to the cable, which acts on any observation point on the body can be estimated.

Specifically, in the arm control system according to the present embodiment, the joint state acquisition unit 241 acquires the state of the joint unit 130 of the arm unit 120. Then, the disturbance estimation unit 251 estimates the external force due to the disturbance on the basis of the condition that the external force due to the predetermined disturbance is limited to one predetermined direction or a plurality of predetermined directions, and the state of the joint unit 130. In other words, the disturbance estimation unit 251 estimates the external force due to the disturbance on the basis of the state of the joint unit 130 after limiting the direction of the external force to be detected to the one predetermined direction or the plurality of predetermined directions.

At this time, the external force estimation unit 251 estimates the external force acting on a predetermined observation point. Note that the one predetermined direction or a plurality of predetermined directions in which the external force is limited can include a rotation direction (moment direction) in addition to a translational direction. Furthermore, the arm control system can function as a medical support arm system in a case of being applied to the medical field. Furthermore, hereinafter, a hard endoscope will be mainly described as an example of an endoscope, but a soft endoscope may be used instead of the hard endoscope.

More specifically, according to the technology of the present embodiment, there are effects such as “(2-1) the observation point is placed on the camera head, and the external force can be perceived as human operation force”, “(2-2) the observation point is placed at the distal end of the hard endoscope, and a contact collision of the distal end of the hard endoscope can be detected”, “(2-3) the observation point is placed at the trocar point, and the force acting from the trocar can be perceived”, and “(2-4) in above (2-1) to (2-3), “(A) by placing restrictions on the perceived disturbance”, “(B) or by giving redundancy to arm degrees of freedom”, “(C) or by installing a force sensor at a specific part of the distal end”, the operation force and the contact and collision can be detected in a complex manner”.

FIG. 9 is a view for describing an example of a force acting from a trocar point. Referring to FIG. 9, a hard endoscope unit 425 is illustrated. Furthermore, a trocar point 71 at which the hard endoscope unit 425 is inserted into the body cavity of the patient 70 is shown. In the example illustrated in FIG. 9, the external force acting on the hard endoscope unit 425 is constrained by the trocar point 71. More specifically, illustrated in FIG. 9, the external force acting on the hard endoscope unit 425 is limited to a pitch direction, a roll direction, and a zoom direction.

Hereinafter, specific examples will be described. The external torque τₙ observed by the VA installed in each joint unit is expressed as the following (13).

[Math. 12]

τᵢ:i∈[1,n] (13)

Note that, in FIG. 3, the external torques observed by the VA installed in the joint units are represented as τ₁, τ₂, τ₃, . . . , and τₙ. Furthermore, the external forces acting on respective observation points are represented as follows.

f_{cable}: Tension (fx, fy, fz) by the cable

fₒₚ: Tension (fx, fy, fz) by the hand holding the camera head

f_{trocar}: Force (fx, fy) acting from the trocar

fₜᵢₚ: Force (fx, fy, fz) acting on the distal end of the hard endoscope

Note that, in FIG. 3, the external forces acting on the respective observation points are represented as f_{cable}, fₒₚ, f_{trocar}, . . . , and fₜᵢₚ. Furthermore, a basic expression representing the relationship between the external force and the external torque is expressed as in (14) below.

$$\begin{matrix}[ Math . 13 ] & \\ {τ}^{T} = {{[ \begin{matrix}{J}_{tip} \\ {J}_{trocar} \\ {J}_{op} \\ {J}_{cable}\end{matrix} ]}^{T} [ \begin{matrix}{J}_{tip} {J}_{trocar} & {J}_{op} {J}_{cable}\end{matrix} ]}^{T} & ( 14 )\end{matrix}$$

Note that, to measure all the forces, torque values for eleven axes are required. Furthermore, in a case of using a moment as an operation force, torque values for a maximum of fourteen axes are required. Furthermore, full force sense can be detected with a configuration of eight-axis redundant degrees of freedom arm six-axis force sensor.

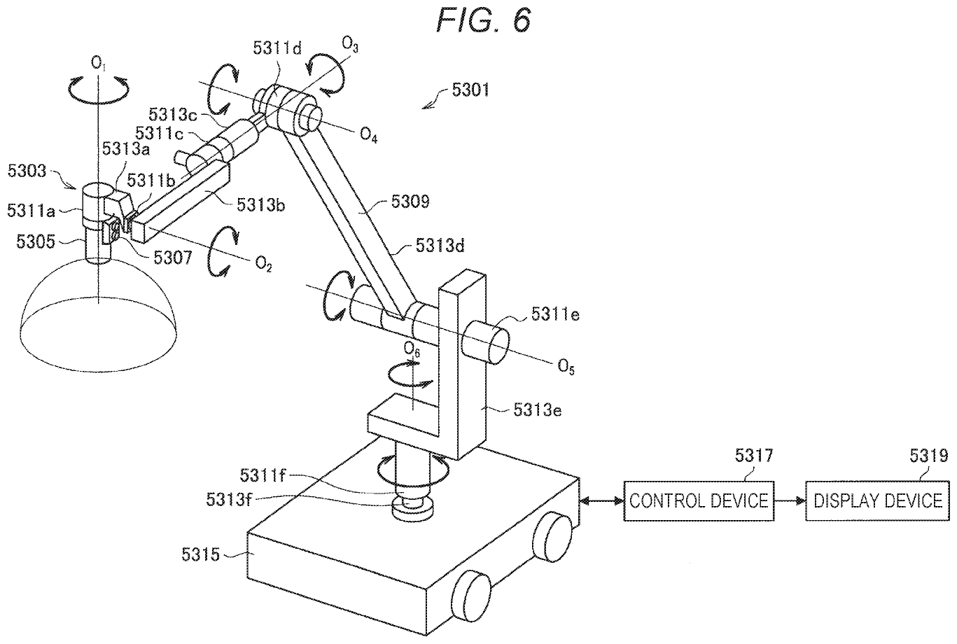

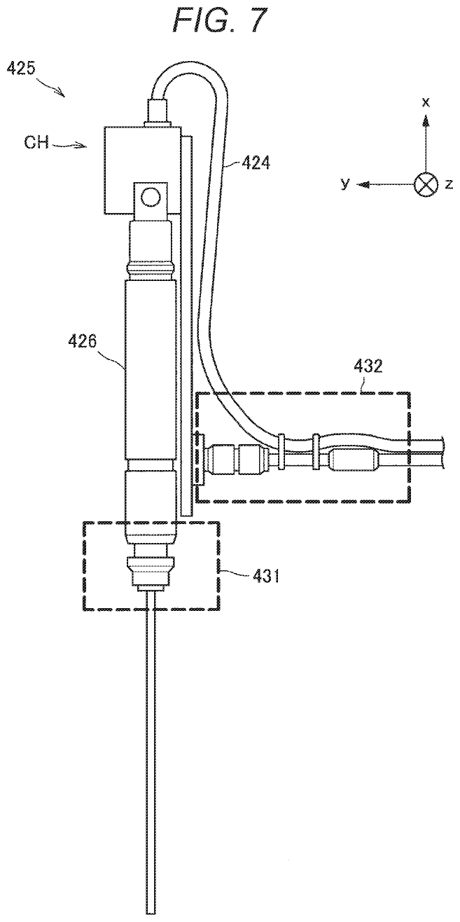

Here, the reason why the tension by the cable is limited will be described. FIG. 7 is a view illustrating an appearance of the hard endoscope unit. As illustrated in FIG. 7, the hard endoscope unit 425 includes a hard endoscope main body 426, a cable 424, and a connection portion 432. The hard endoscope main body 426 includes a camera head CH. Furthermore, the hard endoscope main body 426 includes a holding portion 431 held by the arm. The connection portion 432 is a connection portion between the hard endoscope main body 426 and the cable 424.

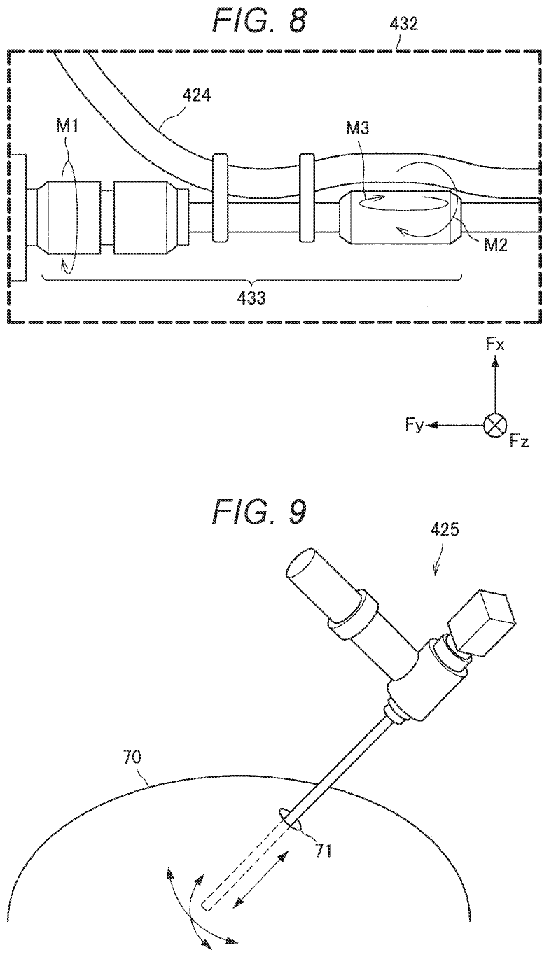

FIG. 8 is an enlarged view of the connection portion 432. The connection portion 432 includes a cable connection component 433 for connecting the hard endoscope main body 426 and the cable 424. The cable connection component 433 is a rigid body. Here, the value of the external moment at the connection portion 432 is considered to be extremely smaller than other types of disturbance, for the reasons described below.

Reason 1: The connection portion between the cable connection component 433 and the hard endoscope main body 426 is designed to be free (to make friction small) with respect to rotation in a direction M1.

Reason 2: The moment arm is extremely short (for example, at most about 5 [mm] equal to the radius of the cable 424) in the connection portion between the cable connection component 433 and the cable 424.

In light of the foregoing, the disturbance acting on the hard endoscope unit 425 from the cable 424 can be regarded as a force in a triaxial horizontal direction. Note that FIG. 8 illustrates directions in which the moment generated by the disturbance is considered to be small due to structural reasons (the direction M1, a direction M2, and a direction M3).

From the formulation of disturbance estimation using the selection matrix described in (14), the following (15) is established.

$$\begin{matrix} [ Math . 14 ] & \\ {[ \begin{matrix}{f}_{tip} {f}_{trocar} & {f}_{op} {f}_{cable}\end{matrix} ]}^{T} [ \begin{matrix}{k}_{1} & … & 0 \\ ⋮ & ⋱ & ⋮ \\ 0 & … & {k}_{x}\end{matrix} ] = {( {[ \begin{matrix}{J}_{tip} \\ {J}_{trocar} \\ {J}_{op} \\ {J}_{cable}\end{matrix} ]}^{T} [ \begin{matrix}{k}_{1} & … & 0 \\ ⋮ & ⋱ & ⋮ \\ 0 & … & {k}_{x}\end{matrix} ] )}^{\#} {τ}^{T} & ( 15 )\end{matrix}$$

Here, in a case where the arm is an endoscope arm that supports an endoscope device, the disturbance estimation unit 251 can estimate the disturbance by the following (16) at the time of an assist operation.

$$\begin{matrix} [ Math . 15 ] & \\ {[ {f}_{op} {f}_{cable} ]}^{T} [ \begin{matrix}{k}_{1} & … & 0 \\ ⋮ & ⋱ & ⋮ \\ 0 & … & {k}_{x}\end{matrix} ] = [ \begin{matrix}[ 0 ] & & & 0 \\ & [ 0 ] & & \\ & & [ 1 ] & \\ 0 & & & [ 1 ]\end{matrix} ] & ( 16 )\end{matrix}$$

Furthermore, in a case where the arm is an endoscope arm that supports an endoscope device, the disturbance estimation unit 251 can estimate the disturbance by the following (17) at the time of an assist operation.

$$\begin{matrix} [ Math . 16 ] & \\ {[ \begin{matrix}{f}_{tip} & {f}_{cable}\end{matrix} ]}^{T} [ \begin{matrix}{k}_{1} & … & 0 \\ ⋮ & ⋱ & ⋮ \\ 0 & … & {k}_{x}\end{matrix} ] = [ \begin{matrix}[ 1 ] & & & 0 \\ & [ 0 ] & & \\ & & [ 0 ] & \\ 0 & & & [ 1 ]\end{matrix} ] & ( 17 )\end{matrix}$$

Furthermore, in a case where the arm is a camera arm supporting a camera (for example, a microscope unit), the disturbance estimation unit 251 can estimate a force received from a monitor that displays navigation information and a force operated by a person by the following (18) at the time of an assist operation.

$$\begin{matrix} [ Math . 17 ] & \\ {[ \begin{matrix}{f}_{tip} & {f}_{op}\end{matrix} ]}^{T} [ \begin{matrix}{k}_{1} & … & 0 \\ ⋮ & ⋱ & ⋮ \\ 0 & … & {k}_{x}\end{matrix} ] = [ \begin{matrix}[ 1 ] & & & 0 \\ & [ 0 ] & & \\ & & [ 1 ] & \\ 0 & & & [ 0 ]\end{matrix} ] & ( 18 )\end{matrix}$$

Furthermore, in a case where the arm is a camera arm supporting a camera (for example, a microscope unit), the disturbance estimation unit 251 can estimate a force received from a monitor that displays navigation information by the following (19) at the time of a remote operation.

$$\begin{matrix} [ Math . 18 ] & \\ {[ {f}_{tip} ]}^{T} [ \begin{matrix}{k}_{1} & … & 0 \\ ⋮ & ⋱ & ⋮ \\ 0 & … & {k}_{x}\end{matrix} ] = [ \begin{matrix}[ 1 ] & & & 0 \\ & [ 0 ] & & \\ & & [ 0 ] & \\ 0 & & & [ 0 ]\end{matrix} ] & ( 19 )\end{matrix}$$

Note that a surgical navigation system may be connected to the camera arm as an external device. In a case of introducing a navigation system, a monitor for navigation and the like are installed (connected) to the arm. With the installation, the arm's own weight (including physical data) deviates from a design value and a negative effect on force control is predicted. By estimating the force received from the monitor as an external force according to this idea, change from the designed value of the arm's own weight can be compensated. Note that the surgical navigation system may also be included in the medical support arm system according to the present embodiment.

Furthermore, in a case where the arm is an arm supporting a retractor or a forceps, the disturbance estimation unit 251 can estimate a force received from the monitor that displays navigation information and a force of the person (operator) who operates the retractor or the forceps by the following (20) at the time of an assist operation, using fₒₚ as the force of the operator who operates the retractor, the forceps, or the like.

$$\begin{matrix} [ Math . 19 ] & \\ {[ \begin{matrix}{f}_{tip} & {f}_{op}\end{matrix} ]}^{T} [ \begin{matrix}{k}_{1} & … & 0 \\ ⋮ & ⋱ & ⋮ \\ 0 & … & {k}_{x}\end{matrix} ] = [ \begin{matrix}[ 1 ] & & & 0 \\ & [ 0 ] & & \\ & & [ 1 ] & \\ 0 & & & [ 0 ]\end{matrix} ] & ( 20 )\end{matrix}$$

Furthermore, in a case where the arm is an arm supporting a retractor or a forceps, the disturbance estimation unit 251 can estimate a force received from a monitor that displays navigation information by the following (21) at the time of a remote operation.

$$\begin{matrix} [ Math . 20 ] & \\ {[ {f}_{tip} ]}^{T} [ \begin{matrix}{k}_{1} & … & 0 \\ ⋮ & ⋱ & ⋮ \\ 0 & … & {k}_{x}\end{matrix} ] = [ \begin{matrix}[ 1 ] & & & 0 \\ & [ 0 ] & & \\ & & [ 0 ] & \\ 0 & & & [ 0 ]\end{matrix} ] & ( 21 )\end{matrix}$$

Note that, when it is considered that the force acting on the distal end is narrowed to a translational force in view of the fact that the distance (moment arm) from an acting point to the observation point is long, the system cannot be an underestimation system. Here, the underestimation system refers to a system in which the number of unknown variables to be estimated exceeds the number of measurable variables, and the values of the unknown variables cannot be uniquely determined (estimated).

The specific disturbance estimation has been described above.

### 5. Joint Control According to External Force According to Present Embodiment

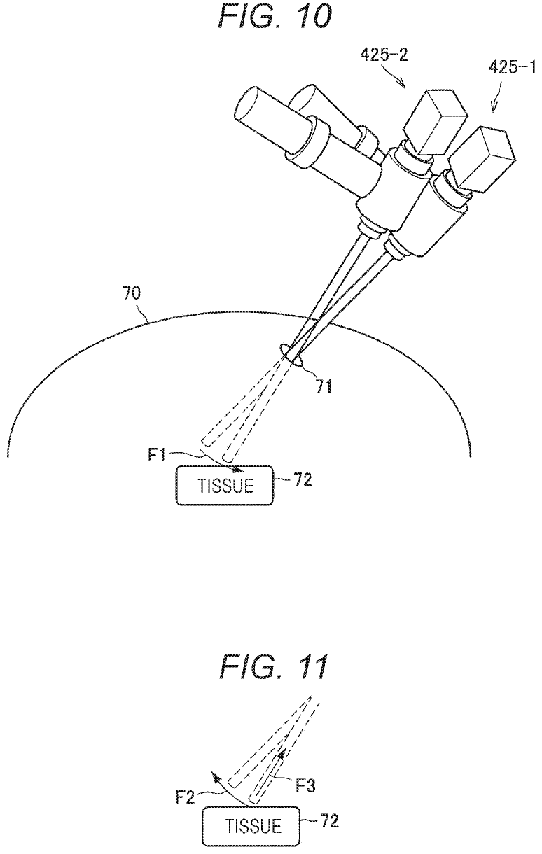

When the external force is estimated by the disturbance estimation unit 251, the command value calculation unit 252 controls the joint unit 130 according to the estimated external force. The command value calculation unit 252 can function as a joint control unit. For example, in a case where the observation point is placed on the distal end of the hard endoscope, the disturbance estimation unit 251 estimates the external force acting on the distal end of the hard endoscope, and the command value calculation unit 252 controls the joint unit 130 according to the external force. Such an example will be described with reference to FIGS. 10 and 11.

FIGS. 10 and 11 are views for describing an example of joint control in a case where an observation point is placed at a distal end of the hard endoscope. Referring to FIG. 10, a hard endoscope 425-1 is moved to a hard endoscope 425-2 by the arm unit 120. In the example illustrated in FIG. 10, an external force F1 acts on the distal end of the hard endoscope. Here, assuming a case in which the distal end of the hard endoscope interferes with a tissue 72 of a patient 70, as illustrated in FIG. 11, an external force F2 and an external force F3 acting on the distal end of the hard endoscope from the tissue 72 are estimated by the disturbance estimation unit 251.

At this time, the command value calculation unit 252 controls the joint unit 130 such that the arm unit 120 moves in a direction according to a direction of the external force F2 or the external force F3, or the arm unit 120 stops. With the control, even in a case where the hard endoscope is mistakenly operated and the distal end of the hard endoscope is brought in contact with the tissue 72 to harm the patient, the external force acting on the distal end of the hard endoscope is recognized, and the arm unit 120 is stopped or avoided to a safe direction, for example, whereby the safety at the time of surgery can be increased. The direction according to the direction of the external force F2 or the external force F3 may be the same as the external force F2, or may be the direction according to the direction of the external force F3.

Meanwhile, for example, in a case where the observation point is placed on the camera head, the disturbance estimation unit 251 estimates the external force acting on the camera head, and the command value calculation unit 252 controls the joint unit 130 according to the external force. Specifically, in a case where it is estimated that an external force acts on the camera head, the command value calculation unit 252 controls the joint unit 130 such that the arm unit 120 moves in a direction according to the direction of the external force. The direction according to the direction of the external force may be the same direction as the external force. In doing so, the arm unit 120 is moved in the direction intended by the operator.

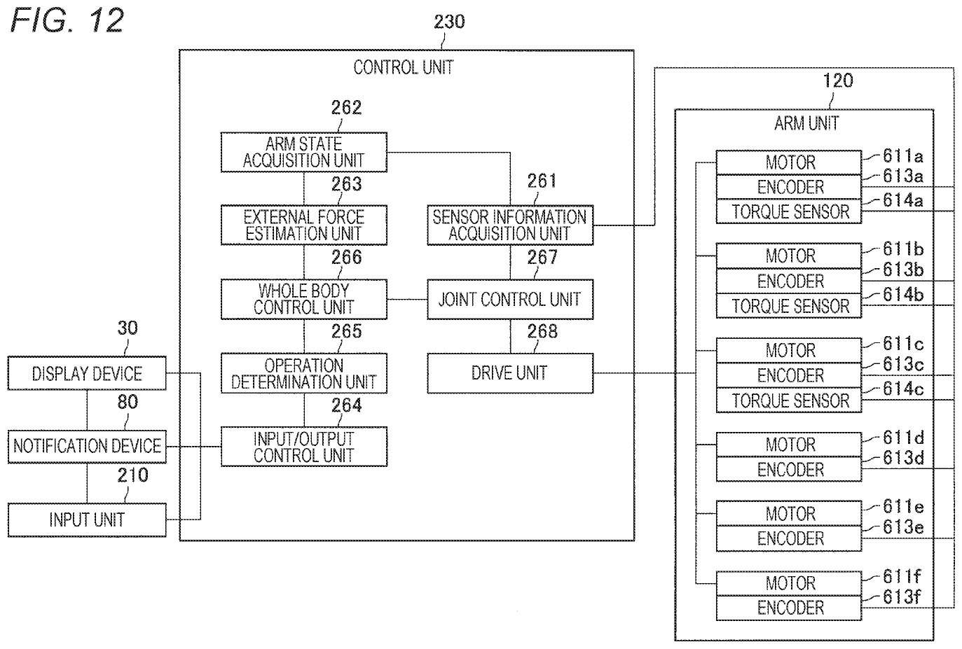

It is difficult for the operator (surgeon) to receive a sense of force or touch in a holding device of a master slave or remote control endoscope. Therefore, the operator may recognize that the hard endoscope is mistakenly giving a tissue a stress by an alert, so that the operation can safely continue the surgery. Specifically, an output control unit 264 (FIG. 12) may control the alert to be output by an output device in a case where the external force exceeds a threshold value. Alternatively, the output control unit 264 (FIG. 12) may perform control such that the magnitude of the external force or the direction of the external force is output by the output device.

Here, the output device may be the display device 30 that performs display so as to be visually perceived by the operator, or may be a notification device 80 (FIG. 12). The notification device 80 includes at least one of a sound output device (such as a buzzer) that outputs a sound so that the operator or the surrounding medical staff audibly perceives the sound by the operator, or a light output device (such as a lamp) that outputs light. Furthermore, the alert may be stoppable by a stop instruction input via the input unit 210.

The joint control according to the specific external force has been described above.

### 6. Specific Configuration Example of Arm Control System

Next, a specific configuration example of the arm control system will be described. FIG. 12 is a diagram illustrating a specific configuration example of the arm control system. As illustrated in FIG. 12, the arm control system includes the arm unit 120, the control unit 230, the input unit 210, the display device 30, and a notification device 80. The functions of the input unit 210, the display device 30, and the notification device 80 are as described above. Furthermore, the control unit 230 includes a sensor information acquisition unit 261, an arm state acquisition unit 262, an external force estimation unit 263, an input/output control unit 264, an operation determination unit 265, a whole body control unit 266, a joint control unit 267, and a drive unit 268.

The sensor information acquisition unit 261 acquires the state of each joint (sensor information of the encoder and the torque sensor) of the arm unit 120, and outputs the state to the joint control unit 267 and the arm state acquisition unit 241. The arm state acquisition unit 262 can correspond to the arm state acquisition unit 241 illustrated in FIG. 5. The external force estimation unit 263 can correspond to the disturbance estimation unit 251 illustrated in FIG. 5. The input/output control unit 264 has a function to acquire input information from the input unit 210, and a function to control outputs of output information by the display device 30 and the notification device. The operation determination unit 265 can correspond to the arithmetic condition setting unit 242 illustrated in FIG. 5. The whole body control unit 266 can correspond to the virtual force calculation unit 243 and the real force calculation unit 244 illustrated in FIG. 5. The joint control unit 267 can correspond to the command value calculation unit 252 illustrated in FIG. 5. The drive unit 268 can correspond to the drive control unit 111 illustrated in FIG. 5.

According to the present embodiment, the direction of the external force detected by the disturbance estimation unit 251 is limited to one predetermined direction or a plurality of predetermined directions. Thereby, the number of sensors can be reduced, and cost reduction can be performed. Moreover, in a case where the arm control system is applied to the medical field, the sensor is omitted in an area (area close to the clean area close to the distal end of the arm) overlapping a working area of the operator (surgeon), and a simple structure can be realized.

For example, according to the present embodiment, the number of torque sensors provided in the arm unit 120 can be made smaller than the number of joint units. In the example illustrated in FIG. 12, the arm unit 120 is provided with six joint units, but the torque sensors are provided in only three joint units out of the six joint units (torque sensors 614a to 614c).

Meanwhile, the encoders are provided in all the six joint units (encoders 613a to 613f). As such, the arm unit 120 may include an encoder having six degrees of freedom or an encoder having a degree of freedom larger than six degrees of freedom. Furthermore, the motors are also provided in all the six joint units (motors 611a to 611f).

Furthermore, the arm unit 120 according to the present embodiment includes at least three or more series of joint units, and torque sensors included in adjacent joint units of the three or more joint units have independent degrees of freedom of each other. The torque sensors included in adjacent joint units having independent degrees of freedom of each other means that the rotation directions of the torque sensors of the adjacent joint units are not both the roll direction, the pitch direction, or the yaw direction. By configuring the torque sensors of the adjacent joint units of the three or more joint units to have independent degrees of freedom of each other, the number of torque sensors provided in the arm unit 120 can be reduced to the number smaller than the number of joint units.

Specifically, in a case where the rotation directions of the torque sensors included the three or more joint units are “pitch, roll, yaw”, “roll, yaw, roll”, “pitch, roll, pitch”, or the like, the torque sensors included in the adjacent joint units have independent degrees of freedom of each other. Meanwhile, in a case where the rotation directions of the torque sensors included the three or more joint units are “pitch, pitch, yaw”, or the like, the torque sensors included in the adjacent joint units do not have independent degrees of freedom of each other. In the example illustrated in FIG. 3, in the case of “421c, 421d, 421e”, “421d, 421e, 421f”, or the like, the torque sensors included in the adjacent joint units have independent degrees of freedom of each other.

The specific configuration example of the arm control system has been described above.

According to the present embodiment, provided is a medical support arm system including a joint state acquisition unit configured to acquire a state of a joint unit of an arm unit, and an external force estimation unit configured to estimate an external force due to predetermined disturbance on the basis of a condition that the external force due to the predetermined disturbance is limited to one predetermined direction or a plurality of predetermined directions, and a state of the joint unit.

According to the configuration, in an endoscope holder arm, disturbance due to tension of a camera and a light source cable can be compensated, and more robust control can be realized. Furthermore, according to such a configuration, an operation force by a person or contact with the outside is detected other than the disturbance due to tension of a camera and a light source cable, and the contact detection can be used as an operation trigger.

Although the favorable embodiments of the present disclosure have been described in detail with reference to the accompanying drawings, the technical scope of the present disclosure is not limited to such examples. It is obvious that persons having ordinary knowledge in the technical field of the present disclosure can conceive various modifications or alterations within the scope of the technical idea described in the claims, and the modifications and alterations are naturally understood to belong to the technical scope of the present disclosure.

Furthermore, the effects described in the present specification are merely illustrative or exemplary and are not restrictive. That is, the technology according to the present disclosure can exhibit other effects obvious to those skilled in the art from the description of the present specification together with or in place of the above-described effects.

Note that following configurations also belong to the technical scope of the present disclosure.

(1)

A medical support arm system including:

a joint state acquisition unit configured to acquire a state of a joint unit of an arm unit; and

an external force estimation unit configured to estimate an external force due to predetermined disturbance on the basis of a condition that the external force due to the predetermined disturbance is limited to one predetermined direction or a plurality of predetermined directions, and a state of the joint unit.

(2)

The medical support arm system according to (1), in which

the arm unit includes a number of torque sensors, the number being smaller than the number of joint units configuring the arm unit.

(3)

The medical support arm system according to (1) or (2), in which

the arm unit includes at least three or more series of joint units, and torque sensors included in adjacent joint units of the three or more joint units have independent degrees of freedom of each other.

(4)

The medical support arm system according to any one of (1) to (3), in which

the arm unit includes an encoder having six degrees or more of freedom.

(5)

The medical support arm system according to any one of (1) to (4), in which

the plurality of joint units configuring the arm unit includes a joint unit including an actuator and an encoder, and a joint unit including an actuator, an encoder, and a torque sensor.

(6)

The medical support arm system according to any one of (1) to (5), in which

the external force estimation unit estimates the external force that acts on a predetermined observation point.

(7)

The medical support arm system according to (6), in which

the observation point includes at least one of a trocar point, a camera head, or a distal end of an endoscope.

(8)

The medical support arm system according to (7), in which

the external force estimation unit estimates the external force acting on the observation point, and

the medical support arm system further includes a joint control unit configured to control the joint unit according to the external force.

(9)

The medical support arm system according to (8), in which

the observation point includes the distal end of the endoscope,

the external force estimation unit estimates the external force acting on the distal end of the endoscope, and

the joint control unit controls the joint unit according to the external force.

(10)

The medical support arm system according to (9), in which

the joint control unit controls the joint unit such that the arm unit moves in a direction according to a direction of the external force or the arm unit stops in a case where it is estimated that the external force has acted on the distal end of the endoscope.

(11)

The medical support arm system according to (8), in which

the observation point includes the camera head,

the external force estimation unit estimates the external force acting on the camera head, and

the joint control unit controls the joint unit according to the external force.

(12)

The medical support arm system according to (11), in which

the joint control unit controls the joint unit such that the arm unit moves in a direction according to a direction of the external force in a case where it is estimated that the external force has acted on the camera head.

(13)

The medical support arm system according to any one of (1) to (12), further including:

an output control unit configured to perform control such that an alert is output by an output device in a case where the external force exceeds a threshold value.

(14)

The medical support arm system according to any one of (1) to (12), further including:

an output control unit configured to perform control such that magnitude of the external force or a direction of the external force is output by an output device.

(15)

The medical support arm system according to (13) or (14), in which

the output device includes at least one of a display device, a sound output device, or a light output device.

(16)

The medical support arm system according to any one of (13) to (15), further including:

the output device.

(17)

The medical support arm system according to any one of (1) to (16), in which

the disturbance includes disturbance due to tension of a light source and a camera cable.

(18)

The medical support arm system according to any one of (1) to (17), in which

a monitor included in a navigation system is connected to the arm unit, and

the external force estimation unit estimates a force received from the monitor as the external force.

(19)

The medical support arm system according to (18), further including:

the monitor.

(20)

The medical support arm system according to any one of (1) to (17), in which

the arm unit supports a retractor or a forceps, and

the external force estimation unit estimates a force by which the retractor or the forceps is operated by an operator, as the external force.

(21)

A control device including:

a joint state acquisition unit configured to acquire a state of a joint unit of an arm unit; and

an external force estimation unit configured to estimate an external force due to predetermined disturbance on the basis of a condition that the external force due to the predetermined disturbance is limited to one predetermined direction or a plurality of predetermined directions, and a state of the joint unit.

### REFERENCE SIGNS LIST

1 Arm control system
10 Arm device
20 Control device
30 Display device
110 Arm control unit
111 Drive control unit
120 Arm unit
130 Joint unit
131 Joint drive unit
132 Indirect state detection unit
133 Rotation angle detection unit
134 Torque detection unit
140 Imaging unit
210 Input unit
220 Storage unit
230 Control unit
240 Whole body coordination control unit
241 Arm state acquisition unit
242 Arithmetic condition setting unit
243 Virtual force calculation unit
244 Real force calculation unit
250 Ideal joint control unit
251 Disturbance estimation unit
252 Command value calculation unit

1. A medical support arm system comprising:
a joint state acquisition unit configured to acquire a state of a joint unit of an arm unit; and
an external force estimation unit configured to estimate an external force due to predetermined disturbance on a basis of a condition that the external force due to the predetermined disturbance is limited to one predetermined direction or a plurality of predetermined directions, and a state of the joint unit.

2. The medical support arm system according to claim 1, wherein
the arm unit includes a number of torque sensors, the number being smaller than the number of joint units configuring the arm unit.

3. The medical support arm system according to claim 2, wherein
the arm unit includes at least three or more series of joint units, and torque sensors included in adjacent joint units of the three or more joint units have independent degrees of freedom of each other.

4. The medical support arm system according to claim 1, wherein
the arm unit includes an encoder having six degrees or more of freedom.

5. The medical support arm system according to claim 1, wherein
the plurality of joint units configuring the arm unit includes a joint unit including an actuator and an encoder, and a joint unit including an actuator, an encoder, and a torque sensor.

6. The medical support arm system according to claim 1, wherein
the external force estimation unit estimates the external force that acts on a predetermined observation point.

7. The medical support arm system according to claim 6, wherein
the observation point includes at least one of a trocar point, a camera head, or a distal end of an endoscope.

8. The medical support arm system according to claim 7, wherein
the external force estimation unit estimates the external force acting on the observation point, and
the medical support arm system further comprises a joint control unit configured to control the joint unit according to the external force.

9. The medical support arm system according to claim 8, wherein
the observation point includes the distal end of the endoscope,
the external force estimation unit estimates the external force acting on the distal end of the endoscope, and
the joint control unit controls the joint unit according to the external force.

10. The medical support arm system according to claim 9, wherein
the joint control unit controls the joint unit such that the arm unit moves in a direction according to a direction of the external force or the arm unit stops in a case where it is estimated that the external force has acted on the distal end of the endoscope.

11. The medical support arm system according to claim 8, wherein
the observation point includes the camera head,
the external force estimation unit estimates the external force acting on the camera head, and
the joint control unit controls the joint unit according to the external force.

12. The medical support arm system according to claim 11, wherein
the joint control unit controls the joint unit such that the arm unit moves in a direction according to a direction of the external force in a case where it is estimated that the external force has acted on the camera head.

13. The medical support arm system according to claim 1, further comprising:
an output control unit configured to perform control such that an alert is output by an output device in a case where the external force exceeds a threshold value.

14. The medical support arm system according to claim 1, further comprising:
an output control unit configured to perform control such that magnitude of the external force or a direction of the external force is output by an output device.

15. The medical support arm system according to claim 13, wherein
the output device includes at least one of a display device, a sound output device, or a light output device.

16. The medical support arm system according to claim 13, further comprising:
the output device.

17. The medical support arm system according to claim 1, wherein
the disturbance includes disturbance due to tension of a light source and a camera cable.

18. The medical support arm system according to claim 1, wherein
a monitor included in a navigation system is connected to the arm unit, and
the external force estimation unit estimates a force received from the monitor as the external force.

19. The medical support arm system according to claim 18, further comprising:
the monitor.

20. The medical support arm system according to claim 1, wherein
the arm unit supports a retractor or a forceps, and
the external force estimation unit estimates a force by which the retractor or the forceps is operated by an operator, as the external force.

21. A control device comprising:
a joint state acquisition unit configured to acquire a state of a joint unit of an arm unit; and
an external force estimation unit configured to estimate an external force due to predetermined disturbance on a basis of a condition that the external force due to the predetermined disturbance is limited to one predetermined direction or a plurality of predetermined directions, and a state of the joint unit.
